(19) United States
(12) Patent Application Publication
(10) Pub. No.: US 2026/0033242 A1
(43) Pub. Date: Jan. 29, 2026

(54) FLEXIBLE ACOUSTIC SENSOR SYSTEMS

(71) Applicant: QUALCOMM Incorporated, San Diego, CA (US)

(72) Inventors: Jessica Liu STROHMANN, Cupertino, CA (US); Hrishikesh Vijaykumar PANCHAWAGH, Cupertino, CA (US); Kostadin Dimitrov DJORDJEV, Los Gatos, CA (US); Sumit AGRAWAL, Sunnyvale, CA (US)

(21) Appl. No.: 19/091,761

(22) Filed: Mar. 26, 2025

Related U.S. Application Data
(60) Provisional application No. 63/676,834, filed on Jul. 29, 2024.

(51) Int. Cl. H10N 30/30; G01H 11/08; H10K 59/40; H10K 77/10; H10N 30/80; H10N 30/87; H10N 39/00

(52) U.S. Cl. CPC H10N 30/302; G01H 11/08; H10K 59/40; H10K 77/111; H10N 30/802; H10N 30/87; H10N 39/00

(57) ABSTRACT

Flexible sensing apparatus and various configurations of sensor stacks associated therewith are disclosed. In some embodiments, an acoustic sensing system may include: a flexible substrate comprising polyimide and having a thickness between 5 and 80 μm; and a flexible acoustic sensor element disposed adjacent to the flexible substrate, and including a stack of materials, the stack of materials including: an acoustic receiver element configured to detect one or more acoustic signals received through the flexible substrate; a piezoelectric layer disposed adjacent to the acoustic receiver element; and an acoustic transmitter element configured to transmit one or more acoustic signals through the flexible substrate. The flexible substrate and the flexible acoustic sensor element may be configured to conform to a curvature of a surface that is constructed to contact a body part of a user from which the one or more acoustic signals transmitted from the acoustic transmitter element are reflected.

### RELATED APPLICATIONS

This application claims the benefit of U.S. Provisional Application No. 63/676,834, filed Jul. 29, 2024, entitled “FLEXIBLE ACOUSTIC SENSOR SYSTEMS,” which is assigned to the assignee hereof, and incorporated herein in its entirety by reference.

### TECHNICAL FIELD

This disclosure relates generally to devices and systems using acoustic sensing systems.

### DESCRIPTION OF RELATED TECHNOLOGY

A variety of different sensing technologies and algorithms are being implemented in devices. Sensing technology is ubiquitous in devices and can be used in various ways, such as identity and fingerprint detection, and biometric and biomedical applications, including health and wellness monitoring. Biometric authentication via fingerprint sensing is an example of an important feature for controlling access to devices or performing other operations. Some such sensing technologies are, or include, acoustic sensors including ultrasonic sensors. Emerging technologies such as flexible devices, including foldable displays, have demanded sensors that are also flexible. Although some previously deployed devices can provide acceptable results, improved applicability of sensing and detection systems in flexible devices would be desirable.

### SUMMARY

The systems, methods and devices of this disclosure each have several aspects, no single one of which is solely responsible for the desirable attributes disclosed herein.

In one aspect of the present disclosure, an acoustic sensing apparatus is disclosed. In some embodiments, the acoustic sensing apparatus may include: a flexible substrate comprising polyimide and having a thickness between 5 and 80 μm; and a flexible acoustic sensor element disposed adjacent to the flexible substrate, the flexible acoustic sensor element including a stack of materials, the stack of materials including: an acoustic receiver element configured to detect one or more acoustic signals received through the flexible substrate; a piezoelectric layer disposed adjacent to the acoustic receiver element; and an acoustic transmitter element configured to transmit one or more acoustic signals through the flexible substrate.

In some implementations thereof, the flexible substrate and the flexible acoustic sensor element may be configured to conform to a curvature of a surface that is constructed to contact a body part of a user from which the one or more acoustic signals transmitted from the acoustic transmitter element are reflected.

In some embodiments, the acoustic sensing apparatus may include: a flexible substrate comprising polyimide and having a thickness between 5 and 80 μm; and a flexible acoustic sensor element disposed adjacent to the flexible substrate, the flexible acoustic sensor element including a stack of materials, the stack of materials including: an acoustic receiver element configured to detect one or more acoustic signals received through the flexible substrate; a first piezoelectric layer disposed adjacent to the acoustic receiver element; a first acoustic transmitter element configured to transmit one or more acoustic signals through the flexible substrate; and a second acoustic transmitter element configured to transmit one or more acoustic signals through the flexible substrate.

In some implementations thereof, the flexible substrate and the flexible acoustic sensor element may be configured to conform to a curvature of a surface that is constructed to contact a body part of a user from which the one or more acoustic signals transmitted from the first acoustic transmitter element and the second acoustic transmitter element are reflected.

In another aspect of the present disclosure, a flexible display apparatus is disclosed. In some embodiments, the flexible display apparatus may include: a glass-based or plastic-based cover layer; a light-emitting layer disposed adjacent to the cover layer; and a flexible acoustic sensing element including: a polyimide substrate; an acoustic receiver element configured to detect one or more acoustic signals received through the polyimide substrate and the light-emitting layer; and an acoustic transmitter element configured to transmit one or more acoustic signals through the polyimide substrate and the light-emitting layer.

In some implementations thereof, the flexible acoustic sensing element and the flexible display apparatus may be configured to collectively deform such that at least two planes associated with the flexible display apparatus intersect one another during a deformed state of the device.

Details of one or more implementations of the subject matter described in this disclosure are set forth in the accompanying drawings and the description below. Other features, aspects, and advantages will become apparent from the description, the drawings and the claims. Note that the relative dimensions of the following figures may not be drawn to scale.

### BRIEF DESCRIPTION OF THE DRAWINGS

FIG. 1 shows a block diagram that shows example components of an apparatus according to some embodiments described herein.

FIG. 1A shows a block diagram that shows example components of the apparatus of FIG. 1 according to some embodiments.

FIG. 2A shows a block diagram representation of components of an example sensing system.

FIG. 2B shows a block diagram representation of components of an example mobile device that includes the sensing system of FIG. 2A.

FIG. 3A shows a side view of an example configuration of an ultrasonic sensor array capable of ultrasonic imaging.

FIG. 3B shows an example configuration of ultrasonic sensor array.

FIG. 4 is a cross-sectional diagram of an example stack of materials usable with embodiments of the flexible acoustic sensor system disclosed herein.

FIG. 4A is a cross-sectional diagram of a variation of the example stack of materials usable with embodiments of the flexible acoustic sensor system disclosed herein.

FIG. 5 is a cross-sectional diagram of another example stack of materials usable with embodiments of the flexible acoustic sensor system disclosed herein.

FIG. 6 is a cross-sectional diagram of another example stack of materials usable with embodiments of the flexible acoustic sensor system disclosed herein.

FIG. 7 is a cross-sectional diagram of an example implementation of a sensor stack in a device having a display apparatus.

FIG. 8 is a cross-sectional diagram of another example implementation of a sensor stack in a device having a display apparatus, according to some embodiments.

FIG. 9 is a cross-sectional diagram of an example implementation of a sensor stack incorporated with a display apparatus, according to some embodiments.

FIG. 10 is a cross-sectional diagram of another example implementation of a sensor stack incorporated with a display apparatus, according to some embodiments.

FIG. 11 is a cross-sectional diagram of a flexible acoustic sensor system using a curved surface, according to some embodiments

FIG. 12A illustrates an example layout of a wearable device having a sensor apparatus having a region having one or more sensor stacks of the type described herein, according to some embodiments.

FIG. 12B illustrates a simplified diagram of the region having one or more sensor stacks, shown in FIG. 12A, according to some embodiments.

FIG. 13 illustrates a cross-sectional profile of an example target object of a user during a pressure wave experienced by the example target object.

FIG. 14 is a cross-sectional diagram of another example implementation of a sensor stack in a wearable device, according to some embodiments.

FIG. 15 representationally depicts aspects of an example two-dimensional array of sensor elements capable of acoustic (e.g., ultrasonic) signaling and detection.

FIGS. 16A-16C illustrate example arrays of sensor elements with driving schemes applied to rows, columns, and both rows and columns.

FIG. 17 is a simplified diagram illustrating two-way beamforming using row-column driving of a transmit beam and a receive beam with a two-dimensional sensor array, according to some approaches.

FIG. 18 shows an example of an apparatus that is configured to perform a receiver-side beamforming process.

FIG. 19A shows an example sensor element array of the type disclosed herein.

FIG. 19B shows an example sensor element array having defined subarrays of sensor elements.

FIG. 19C shows an example sensor element array having sensor element height differentiation.

FIG. 20 shows an example of a blood pressure monitoring device based on photoacoustic plethysmography, which may be referred to herein as PAPG.

FIG. 21 shows an example of a blood pressure monitoring device based on photoplethysmography (PPG).

FIG. 22 is a cross-sectional diagram of an example implementation of a sensor apparatus with photoacoustics capability, according to some embodiments.

FIG. 23 is a cross-sectional diagram of another example implementation of a sensor apparatus with photoacoustics capability, according to some embodiments.

FIGS. 24A and 24B are block diagrams of example system configurations of a sensor stack.

FIG. 25 shows a flow diagram of an example method of operating a flexible acoustic sensor, according to some embodiments.

FIG. 26 shows a flow diagram of another example method of operating a flexible acoustic sensor, according to some embodiments.

FIG. 27 shows a flow diagram of another example method of operating a flexible acoustic sensor, according to some embodiments.

Like reference numbers and designations in the various drawings indicate like elements.

### DETAILED DESCRIPTION

The following description is directed to certain implementations for the purposes of describing various aspects of this disclosure. However, a person having ordinary skill in the art will readily recognize that the teachings herein can be applied in a multitude of different ways. Some of the concepts and examples provided in this disclosure are especially applicable to user sensing applications. For example, fingerprint detection can be performed using the disclosed embodiments. However, some implementations also may be applicable to other types of sensing applications including biometric sensing, as well as to various other systems. The described implementations may be implemented in any device, apparatus, or system that includes an apparatus as disclosed herein. In addition, it is contemplated that the described implementations may be included in or associated with a variety of electronic devices (which may also be referred to herein simply as “devices” or a “device”) such as, but not limited to, mobile telephones, multimedia Internet-enabled cellular telephones, mobile television receivers, wireless devices, smartphones, smart cards, tablets, wearable devices such as bracelets, armbands, wristbands, watches, smartwatches, rings, headbands, patches, chest bands, anklets, etc., Bluetooth® devices, personal data assistants (PDAs), wireless electronic mail receivers, handheld or portable computers, netbooks, notebooks, smartbooks, printers, copiers, scanners, facsimile devices, global positioning system (GPS) receivers or navigators, cameras, digital media players, game consoles, wrist watches, clocks, calculators, television monitors, flat panel displays, electronic reading devices (e.g., e-readers), mobile health devices, computer monitors, auto displays (including odometer and speedometer displays, dashboard displays, etc.), cockpit controls and/or displays, camera view displays (such as the display of a rear view camera in a vehicle), architectural structures, microwaves, refrigerators, stereo systems, cassette recorders or players, DVD players, CD players, VCRs, radios, portable memory chips, washers, dryers, washer/dryers, parking meters, automobile doors, Internet of Things (IOT) devices, palm scanners, or point-of-sale (POS) terminals. Thus, the teachings are not intended to be limited to the specific implementations depicted and described with reference to the drawings; rather, the teachings have wide applicability as will be readily apparent to persons having ordinary skill in the art.

Modern devices include various functionalities and hardware that support the functionalities. As but one example, fingerprint sensing using a sensor is one such function of a device. In some embodiments, acoustic imaging, e.g., via transmission and receipt of ultrasonic signals by an acoustic transmitter element and an acoustic receiver element of the fingerprint sensor, may be used to obtain the fingerprint data.

As an aside, toe prints can be used to identify users because they are unique and permanent, similar to fingerprints. Toc prints have ridge (raised portions) patterns and furrows (recessed portions, otherwise known as valleys) similar to fingerprints. Similar to fingerprints, toe prints have unique features referred to as minutiae points that can differentiate one person from another. The whorls, ridges, valleys, and furrows in toe prints develop uniquely in each person. Therefore, the embodiments described herein can be used with toes for equal effectiveness as with fingers. Palms and feet may also be used for identification using unique features. However, toes, palms and feet are used less often for identification, particularly with aforementioned types of devices. For simplicity, “fingerprint” in the context of the present disclosure may refer to fingerprints, toe prints, palm prints, or footprints, and “finger” may refer to fingers, toes, palms, or feet.

Fingerprint sensing can be used by software and applications (apps) usable with a device to biometrically authenticate a user. Fingerprint data obtained using a fingerprint sensor may be used by the device to identify an object (such as a finger or fingerprint), change an operative state of the device, and/or perform other operations with the device (unlock or lock the device, initialize an application, authenticate a user, etc.). Some devices may be configured such that the sensor (such as a fingerprint sensor) is disposed beneath a display or other surface, which in cases of some devices (smartphone, tablets, etc.) may be a screen or other user interface.

Fingerprint sensors are thus useful for various purposes and are usable with various types of devices and/or displays. However, there are performance limitations when it comes to certain devices. As one example, flexible or foldable devices, when using typical sensors do not have the level of sensing performance that can be seen with, e.g., flat-panel displays. As a more specific example, ultrasonic signals transmitted or received by conventional sensors in conventional foldable displays or display stacks may have a transmission rate or a signal strength that is as little as 25-35% of that of an OLED (organic light-emitting diode) panel or a plastic OLED (POLED). As acoustic sensing often uses plane-wave propagation, weak signals are a challenge especially in fingerprint sensing with flexible (e.g., foldable) devices. As consumer devices and display technologies continue to mature, and flexible displays become more applicable in existing and emerging technologies, improving the performance of sensors in such flexible devices (which may include or utilize curved surfaces or displays or screens) can improve user experience and allow the sensors to be used with many types of devices and other objects.

In some embodiments described in the present disclosure, an acoustic (e.g., ultrasonic) sensor apparatus or system may include a stack of materials comprising a sensor element and other components that enable propagation and detection of acoustic signals. The sensor apparatus may have physically flexible and pliable properties so as to allow the sensor apparatus to conform to a non-planar surface, such as a curved or rounded surface, or a surface that can be deformed to be curved or rounded along at least one axis. For example, the sensor apparatus may be used with a foldable device or a device having a curved surface or platen. The sensor stack may include materials to enable the flexibility and pliability of the sensor apparatus, such as a flexible substrate composed of polyimide in some embodiments, or other types of polymers in other embodiments.

Various embodiments of the sensor stack are disclosed herein. In some embodiments, the sensor stack may include layer of thin-film transistor (TFT) circuitry grown on a flexible substrate, a piezoelectric layer comprising a copolymer adjacent to the TFT layer, and an electrode layer adjacent to the piezoelectric layer. In some cases, a passivation layer may be disposed adjacent to the electrode layer. In some implementations, the electrode layer may receive transmit signals that cause emission of acoustic (e.g., ultrasonic) waves toward a target object of interest (e.g., a finger at a surface of a platen on the other side of the flexible substrate). The TFT circuitry may include one or more acoustic receiver pixels that receive electric signals generated from the piezoelectric copolymer layer that receives acoustic (e.g., ultrasonic) waves reflected from the object of interest. The passivation layer may include a protective coating, such as ink.

In some other embodiments, the sensor stack may be in an opposite orientation such that the flexible substrate is placed away from the platen. In some other embodiments, at least one additional piezoelectric layer and at least one additional electrode layer may be included. In some embodiments, the sensor stack may be embedded within a display apparatus, such as underneath a cover glass or platen and a light-emitting (e.g., OLED) layer, but above a backplate of the display apparatus.

In some embodiments, a sensor stack may be implemented in a thin and flexible form factor that can be used with various types of surfaces. For example, the form factor may be a patch that may be secured or attached to a user's skin (e.g., at the wrist) to operate as a biosensor.

In addition to acoustic sensing (e.g., with generated and received ultrasonic waves) with the disclosed sensor stacks, further implementations include various sensing modalities in conjunction with the types of sensor stacks disclosed herein. In some such implementations, photoacoustic sensing may be used with a light source system having one or more light sources and/or waveguides. In some cases, a coupling element such as coupling film or coupling mold that is optically and acoustically transparent may be used to allow coupling with a surface, such as skin. In some implementations, piezo-sensing with the piezoelectric layer of the sensor stack, or optical sensing may be used as well. Heart rate waveforms (HRW) and physiological characteristics such as pulse wave velocity (PWV) can be obtained from one or more of the aforementioned modalities.

Disclosed implementations may be capable of capturing information about a target object such as a blood vessel, including cross-sectional area, PWV, and others. Such information can then be used to estimate useful parameters such as blood pressure. Various beamforming techniques can be used to enhance measurements obtained by the sensor stack, including row-column driving, delay-and-sum, subdivision of sensor elements of an array, and sensor element height differentiation.

Particular implementations of the subject matter described in this disclosure can be implemented to realize one or more of the following potential advantages. The configurations disclosed herein may enable acoustic sensors to be utilized in various types of surfaces (e.g., curved or distorted surfaces) and/or flexible devices (e.g., foldable displays) by using a flexible sensor stack, while maintaining reliability and achieving high resolution under display with a very thin stack. By selecting certain layers in the sensor stack, various thicknesses and can be achieved. Usage of polyimide (or another similar acoustically and/or optically transparent polymer) provides the flexibility that allows the sensor apparatus to conform to curved surfaces. The variety of sensor stacks disclosed herein advantageously enable various configurations, such as, for example, larger emitted and returning acoustic signals and increased signal sensitivity.

In addition to implementation with flexible devices (e.g., foldable displays) and fingerprint sensing, the flexible sensor configuration may be thin and flexible enough to easily conform to the surface of the human body (e.g., wrist) and curved surfaces (doorknob, steering wheel, etc.), enabling the sensor stacks disclosed herein to operate as a biosensor with a thin, flexible, and non-invasive form factor, such as a patch. Implementations using an adhesive or coupling layer can also keep the sensor in place without needing external pressure, increasing measurement consistency and reducing measurement complexity. Using one or more beamforming techniques can improve the fidelity and strength of received signals. These tunable configurations allow the disclosed sensor stacks to be used in a variety of applications and use cases, such as wearable devices, devices with curved surfaces, flexible displays, medical devices, ultrasonic patches, and others. Adaptability of the disclosed flexible sensor stacks to being used with various modalities such as acoustic (e.g., ultrasonic), photoacoustic, piezoelectric, and/or optical allow various ways to obtain measurements relating to the body, such as characteristics of blood vessel (e.g., PWV) and useful user parameters such as blood pressure based on PWV in a non-invasive manner. Hence, the present disclosure represents a significant advancement in sensing technology.

Additional details will follow after an initial description of relevant systems and technologies.

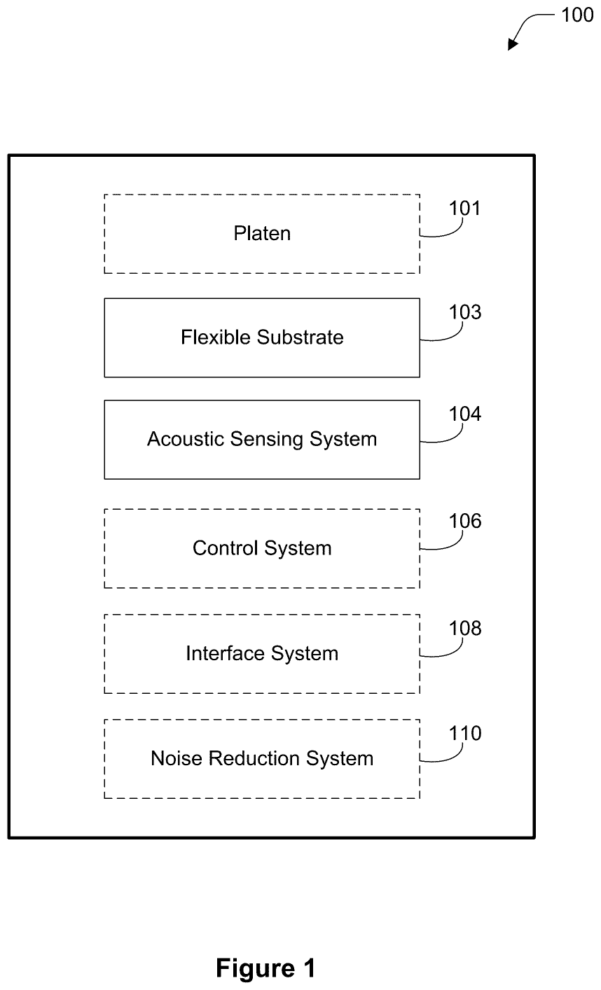

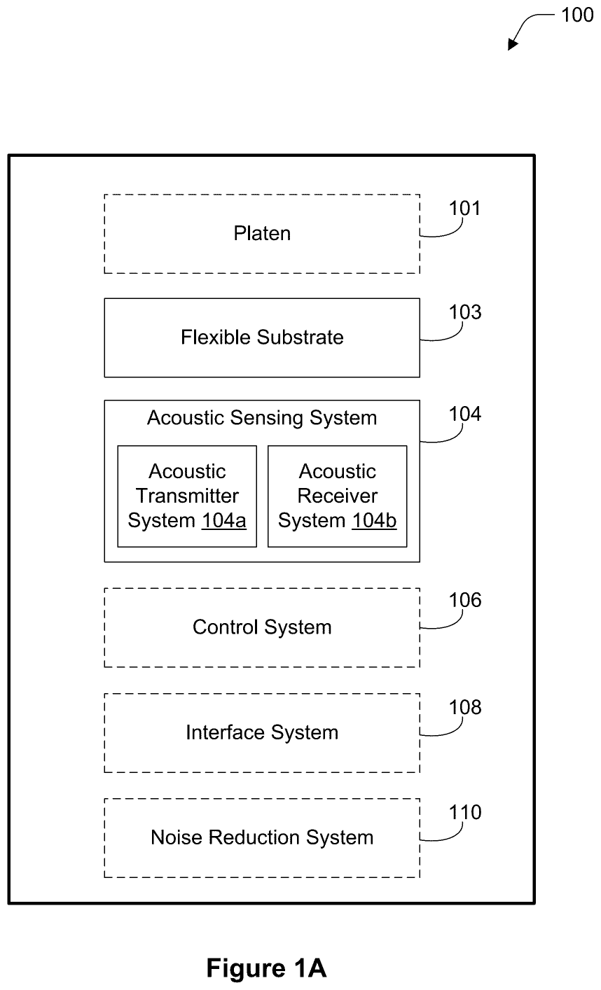

FIG. 1 is a block diagram that shows example components of an apparatus 100 according to some implementations. In some example embodiments, the apparatus 100 may include a flexible substrate 103 and an acoustic sensing system 104.

Some implementations of the apparatus 100 may include a control system 106, an interface system 108, a noise reduction system 110, or a combination thereof.

In some configurations, apparatus 100 may be a sensor, sensor apparatus, or a sensing system usable with an electronic device such as that listed elsewhere above. In some configurations, apparatus 100 may be part of the device or another apparatus.

In some applications, platen 101 may be included with the apparatus 100 or separate from the apparatus 100. Platen 101 may be or include a surface of a device. Some examples of a platen 101 may be part of, or include, a display apparatus, such as an OLED panel or another flat-panel display, or a flexible display, or a layer of a stack of materials of a display apparatus. The platen 101 may at least partly include a visually and/or optically transparent portion.

While platens generally have rigid and inflexible surfaces, the platen 101 disclosed herein may not be so rigid (or may be rigid in some cases, e.g., glass panel). In various implementations, platen 101 may include a surface that is capable of bending, folding, or other distortions, or it may be fixed at, or as, a curved surface. To achieve this flexibility, platen 101 may be composed of a polymer such as polyethylene, parylene, polystyrene, polyurethane rubber, or another flexible material. In further examples, the platen 101 may be a surface of an object such as the handle of a steering wheel of a vehicle (which typically has a curved geometry similar to a torus), a curved edge of a touchscreen, a surface of a mobile device such the side of a headset, a surface of a controller such as a handheld and/or wireless controller for controlling or interacting with extended reality (XR) (including virtual reality (VR), augmented reality (AR), mixed reality (MR), a wristwatch or wristband, a doorknob or handle, a pole or pole-shaped object or device, a wall, an electronic device listed above, or other surfaces of an object or device that may be communicatively and/or physically coupled with an electronic device or other computerized apparatus.

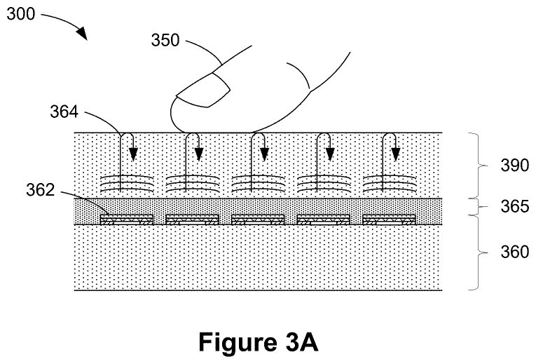

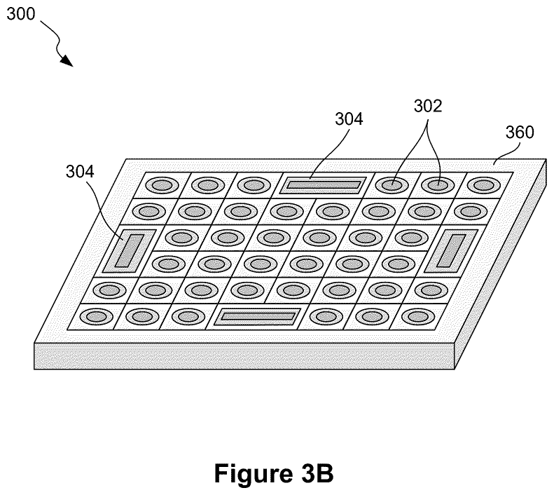

The platen 101 may be constructed such that a portion or a body part of a user (e.g., a finger) can be received by and make contact with the platen 101. In some applications, at least a portion of the platen 101 may be associated with a sensing portion or a sensing area, where acoustic (e.g., ultrasonic) sensing may occur with an object such as a portion or body part of a user (e.g., a finger). Further features of the platen 101 relating to transmission of acoustic signals and receipt of acoustic signals reflected from the portion of the user will be described with respect to platen 390 in FIG. 3A.

As will be described further below, the flexible substrate 103 (and/or other components of the apparatus 100 or the associated stack of materials) may give the apparatus 100 the capability to be curved to conform to any shape, such as the shape of the platen 101 or other desired shape. For instance, during the bending, folding, or twisting of a device implementing the apparatus 100, the apparatus 100 may also be bent, folded, or twisted. As alluded to above, the apparatus 100 may alternatively be fixed to a bent, folded, twisted, or otherwise curved surface.

In some embodiments, the flexible substrate 103 may be disposed adjacent to an acoustic sensing system 104. In some embodiments, an acoustic sensing system 104 may include, e.g., an acoustic transmitter system and an acoustic receiver system, embodiments and implementations of which are described below. The flexible substrate 103 can be conformed to a curved surface (and indeed any shape) because it may be constructed of a flexible material, and thereby allow the apparatus 100 to conform to a curved shape (or deform, e.g., fold or bend). In some implementations, flexible substrate 103 may be a polymer such as polyimide. In other implementations, flexible substrate 103 may be constructed of polyethylene terephthalate (PET), polyethylene naphthalate (PEN), thermoplastic polyurethane (TPU), cellulose paper, polyestersulfone (PES), or colorless polyimide (CPI). In some implementations, flexible substrate 103 may be constructed of stainless steel.

In some implementations, the flexible substrate 103 may have a thickness of 5 to 80 microns (μm). For example, this thickness may be 10 to 50 μm in some examples. Depending on the use case or application, the flexible substrate 103 may have a thickness that is lower or higher than the foregoing range, or on the lower end or the higher end of the foregoing range, to support the desired amount of flexibility. As an illustrative consideration, the flexible substrate 103 may be closer to 10-20 μm thick if more flexibility is desired, e.g., where the apparatus 100 is used with a highly curved surface, or used with a device that folds frequently such as a foldable display. On the other hand, the flexible substrate 103 may be closer to 40-50 μm thick if less flexibility is needed, e.g., where the apparatus 100 is disposed at a substantially planar surface with little curvature. In the case of stainless steel, the thickness may be thinner, e.g., 10-25 μm.

Various configurations of an acoustic transmitter system 104a and an acoustic receiver system 104b are also disclosed herein. As indicated above and in FIG. 1A, acoustic transmitter system 104a and acoustic receiver system 104b may be collectively included in an acoustic sensing element, or the acoustic sensing system 104. For example, acoustic transmitter system 104a and acoustic receiver system 104b may share the same piezoelectric copolymer layer of a stack of materials associated with the apparatus 100. Specific examples of the acoustic transmitter system 104a and the acoustic receiver system 104b are described in more detail below.

In some embodiments, the acoustic transmitter system 104a may be configured to generate and emit acoustic signals, e.g., toward a target object, such as a finger or other object. Acoustic signals may include one or more acoustic waves, such as, in some scenarios, ultrasonic waves 364 as shown in FIG. 3A. In some implementations, the acoustic transmitter system 104a may include one or more ultrasonic transmitters or transmitter elements configured to generate, emit, and/or direct ultrasonic waves. The one or more ultrasonic transmitters may be one or more ultrasonic transducers. In some implementations, ultrasonic waves may be generated in a selected portion of multiple ultrasound transmitter elements (e.g., in an array). In some configurations, the one or more ultrasonic transmitter elements may be arranged in an array of ultrasonic transducer elements, such as an array of PMUTs and/or an array of CMUTs. In some examples, the ultrasonic transmitter(s) may include an ultrasonic plane-wave generator.

In some implementations, a control system 106 may include one or more controllers or processors, or a drive circuit or various types of drive circuitry, configured to control the one or more ultrasonic transmitter elements via one or more instructions to the acoustic transmitter system 104a. For example, ultrasonic waves may be generated in pulses (e.g., at least partly repeating or other patterns) or according to other timing instructions. Although “ultrasound” or “ultrasonic” may typically apply to acoustic energy with a frequency above human hearing, or 20 kilohertz (kHz), ultrasound frequencies used for fingerprint imaging may exceed well over this lower limit. In some implementations, the control system 106 may cause ultrasonic waves from the acoustic transmitter system 104a to be generated and emitted at a frequency that is between about 12 megahertz (MHz) to 50 MHz, which may result in sufficient resolution for fingerprint imaging, e.g., up to 1000 dots per inch (dpi). Other suitable frequencies may be used for the acoustic waves in other implementations.

Control system 106 may be electrically and/or communicatively coupled to the apparatus 100. In some configurations, the control system 106 may be part of the apparatus 100. In some configurations, the control system 106 may be part of a device having the apparatus 100. In some configurations, the control system 106 may be external to the apparatus 100 or the device having the apparatus 100, for example but not limited to, on a server (cloud), remote storage, or another device other than the device having the apparatus 100. In some configurations, the one or more controllers or processors of the control system 106 may be distributed across two or more devices including external apparatus.

In some implementations, the control system 106 may include one or more general purpose single- or multi-chip processors, digital signal processors (DSPs), application-specific integrated circuits (ASICs), field programmable gate arrays (FPGAs) or other programmable logic devices, discrete gates or transistor logic, discrete hardware components, or combinations thereof. The control system 106 also may include (and/or be configured for communication with) one or more memory devices, such as one or more random access memory (RAM) devices, read-only memory (ROM) devices, etc. Accordingly, the apparatus 100 may have a memory system that includes one or more memory devices, though the memory system is not shown in FIG. 1. In some implementations, functionality of the control system 106 may be partitioned between one or more controllers or processors, such as a dedicated sensor controller and an applications processor of a mobile device.

If the apparatus 100 includes an ultrasonic transmitter, such as in the acoustic transmitter system 104a, the control system 106 may be configured for controlling the ultrasonic transmitter. In some embodiments, a control system 106 may cause the acoustic transmitter system 104a to generate and emit acoustic waves. In some implementations, the control system 106 may cause the acoustic transmitter system 104a to generate and emit acoustic waves in response to a detection of an object (e.g., a finger). In some cases, the object may be detected based at least on a force applied to the apparatus 100. Sensor elements 304 may be used for non-ultrasonic force detection, for example. In another example, a resistive sensor or capacitive sensing with a touchscreen may allow detection of sufficient force applied to the apparatus 100.

In some cases, the object may be detected based at least on light occlusion. In such cases, a light sensor may also be included with the apparatus 100 so that an amount of light or its absence (e.g., relative to a threshold) can be determined, e.g., by control system 106, at or near the apparatus 100.

In some cases, the object may be detected based at least on a capacitive shift or response. For example, a capacitive sensor or touchscreen may allow determination of a capacitive response based on the natural conductivity of the object such as a finger that is making contact with the platen 101 of the apparatus 100.

In some implementations, a combination of one or more detection methods described above may be used to detect the object. For instance, detection of the object may require, in some configurations, sufficient force and sufficient capacitive response. In another example, detection of the object may require sufficient force, sufficient capacitive response, and sufficient absence of light.

In some configurations, a delay may be placed between the detection of the object and the emission of the acoustic waves, where the length of the delay may be 100 milliseconds, 500 milliseconds, etc. Not causing emission of acoustic waves immediately may allow time for the object to stabilize against the apparatus 100 before performing, e.g., fingerprint sensing. Force or occlusion may occur even if the finger is not pressed onto the apparatus 100 completely.

In some implementations, the acoustic transmitter system 104a may include one or more acoustic waveguides or ultrasonic waveguides (or other sound-directing elements) constructed to propagate and direct acoustic or ultrasonic waves toward a target location that does not have direct line of sight from at least a portion of the one or more ultrasound transmitter elements. Such waveguides may be useful in certain devices, e.g., foldable displays, or chasses that may optimize the locations of the acoustic transmitter system 104a and the location of a fingerprint sensor by placing them out of direct line of sight.

The acoustic signals (e.g., ultrasonic waves) emitted from acoustic transmitter system 104a may cause or result in reflection of acoustic wave emissions at least in part from the object (e.g., finger). As noted above, characteristics of the reflected waves such as amplitudes may depend in part on the acoustic properties of the object and/or the platen. These reflected acoustic waves (e.g., ultrasonic waves) may be detectable by the acoustic receiver system 104b.

Various examples of an acoustic receiver system 104b are disclosed herein, some of which may include an ultrasonic receiver system. In some implementations, the acoustic receiver system 104b may include an ultrasonic receiver system having the one or more ultrasonic receiver elements. In some implementations, one or more discrete portions, or one or more pixelated receiver electrodes, may form at least part of corresponding one or more acoustic receiver elements represented by one or more receiver pixels, each of which forms part of thin-film transistor (TFT) circuitry. In some implementations, one or more ultrasonic receiver elements and one or more ultrasonic transmitter elements may be combined in an ultrasonic transceiver. In some examples, the acoustic receiver system 104b and the acoustic transmitter system 104a may both include the same piezoelectric receiver layer, such as a layer of polyvinylidene fluoride (PVDF) polymer or a layer of poly(vinylidene fluoride-co-trifluoroethylene) (PVDF-TrFE) copolymer. In some implementations, a single piezoelectric layer may serve as an ultrasonic receiver. In some implementations, other piezoelectric materials may be used in the piezoelectric layer, such as aluminum nitride (AlN) or lead zirconate titanate (PZT). In other implementations, the piezoelectric receiver layer may be composed of ceramics or a single crystal. According to some examples, the acoustic receiver system 104b may be, or may include, an ultrasonic receiver array. The acoustic receiver system 104b may, in some examples, include an array of ultrasonic transducer elements, such as an array of PMUTs, an array of CMUTs, etc. In some such examples, a piezoelectric receiver layer, PMUT elements in a single-layer array of PMUTs, or CMUT elements in a single-layer array of CMUTs, may be used as ultrasonic transmitters (such as those that are included in acoustic transmitter system 104a) as well as ultrasonic receivers. In some examples, the apparatus 100 may include one or more separate ultrasonic transmitter elements or one or more separate arrays of ultrasonic transmitter elements. Ultrasonic sensor array 300, sensor system 202, and ultrasonic sensor array 212 may be examples or implementations of the acoustic receiver system 104b.

In the context of the present disclosure, a transmitter element and a receiver element may collectively or individually be referred to as a “sensing element,” an “acoustic sensing element,” a “sensor element,” or an “acoustic sensor element.” Such an element may also refer to a transceiver element or an acoustic transceiver element. In some instances, the foregoing terms may refer collectively, for example as a sensing element, to a transmitter element and a receiver element that share the same piezoelectric layer.

In some other embodiments, the acoustic receiver system 104b may include one or more microphones configured to detect acoustic signals. Each microphone may be a MEMS (micro-electromechanical system) microphone having an inlet port, a cavity, and/or a membrane or mesh to facilitate detection and receipt of acoustic signals, e.g., sound waves. In some implementations, the microphone(s) may be part of another apparatus or system other than the apparatus 100, such as the interface system 108 described below.

Accordingly, embodiments of apparatus 100 may be configured to operate as ultrasound sensors that are configured to receive reflected acoustic signals such as ultrasonic waves. Reflected ultrasonic waves may include scattered waves, specularly reflected waves, or both scattered waves and specularly reflected waves. The reflected waves can provide acoustic data, including information about the object, e.g., a finger's ridges and valleys and their shapes and patterns.

More specifically, in some embodiments, control system 106 may be configured to receive the acoustic data (e.g., from acoustic receiver system 104b) and/or generate images (e.g., three-dimensional images) representative of the object such as a finger. That is, fingerprint imaging may be performed using the acoustic data received by the acoustic receiver system 104b. Images may be matched to a reference to identify the fingerprint image.

In some examples, the control system 106 may be communicatively coupled to a light source system (not shown) and configured to control the light source system to emit light towards a target object (such as a finger) on an outer surface of the platen 101. In some such examples, the control system 106 may be communicatively coupled to and configured to receive signals from the acoustic receiver system 104b (including one or more receiver elements, such as sensor elements 362) corresponding to the ultrasonic waves generated by the target object responsive to the light from the light source system.

In the context of fingerprint sensing, ultrasonic fingerprint sensing may advantageously be more reliable and secure (e.g., for storing user identifying information), and have a smaller and more flexible footprint, than other types of fingerprint sensing such as traditional optical fingerprint scanning that relies on optical imaging.

Some implementations of the apparatus 100 may include an interface system 108. In some examples, the interface system 108 may include a wireless interface system. In some implementations, the interface system 108 may include a user interface system, one or more network interfaces, one or more communication interfaces between the control system 106 and a memory system and/or one or more interfaces between the control system 106 and one or more external device interfaces (such as ports or applications processors), or combinations thereof. According to some examples in which the interface system 108 is present and includes a user interface system, the user interface system may include a microphone system (including, e.g., one or more microphones), a loudspeaker system, a haptic feedback system, a voice command system, one or more displays, or combinations thereof. According to some examples, the interface system 108 may include a touch sensor system, a gesture sensor system, or a combination thereof. The touch sensor system (if present) may be, or may include, a resistive touch sensor system, a surface capacitive touch sensor system, a projected capacitive touch sensor system, a surface acoustic wave touch sensor system, an infrared touch sensor system, any other suitable type of touch sensor system, or combinations thereof.

In some examples, the interface system 108 may include a force sensor system. The force sensor system (if present) may be, or may include, a piezo-resistive sensor, a capacitive sensor, a thin film sensor (for example, a polymer-based thin film sensor), another type of suitable force sensor, or combinations thereof. If the force sensor system includes a piezo-resistive sensor, the piezo-resistive sensor may include silicon, metal, polysilicon, glass, or combinations thereof. An ultrasonic fingerprint sensor and a force sensor system may, in some implementations, be mechanically coupled. In some implementations, the force sensor system may be mechanically coupled to a platen. In some such examples, the force sensor system may be integrated into circuitry of the ultrasonic fingerprint sensor. In some examples, the interface system 108 may include an optical sensor system, one or more cameras, or a combination thereof.

According to some examples, the apparatus 100 may include a noise reduction system 110. In some implementations, the noise reduction system 110 may include one or more sound-absorbing layers, acoustic isolation material, or combinations thereof. In some examples, the noise reduction system 110 may include acoustic isolation material, which may reside between at least a portion of the acoustic transmitter system 104a and at least a portion of the acoustic receiver system 104b, e.g., between ultrasonic transmitter elements and ultrasonic receiver elements. In some examples, the noise reduction system 110 may include one or more electromagnetically shielded transmission wires. In some such examples, the one or more electromagnetically shielded transmission wires may be configured to reduce electromagnetic interference from circuitry of the acoustic transmitter system 104a, circuitry of the acoustic receiver system 104b, or combinations thereof, that is received by the acoustic receiver system 104b.

In some implementations, the apparatus 100 may be part of a mobile device. In some implementations, the apparatus 100 may be part of a wearable device configured to be worn by a user, such as around the wrist, finger, arm, leg, ankle, or another appendage, or another portion of the body. In an example implementation, the wearable device may have the form of a wristwatch and can be worn around the wrist.

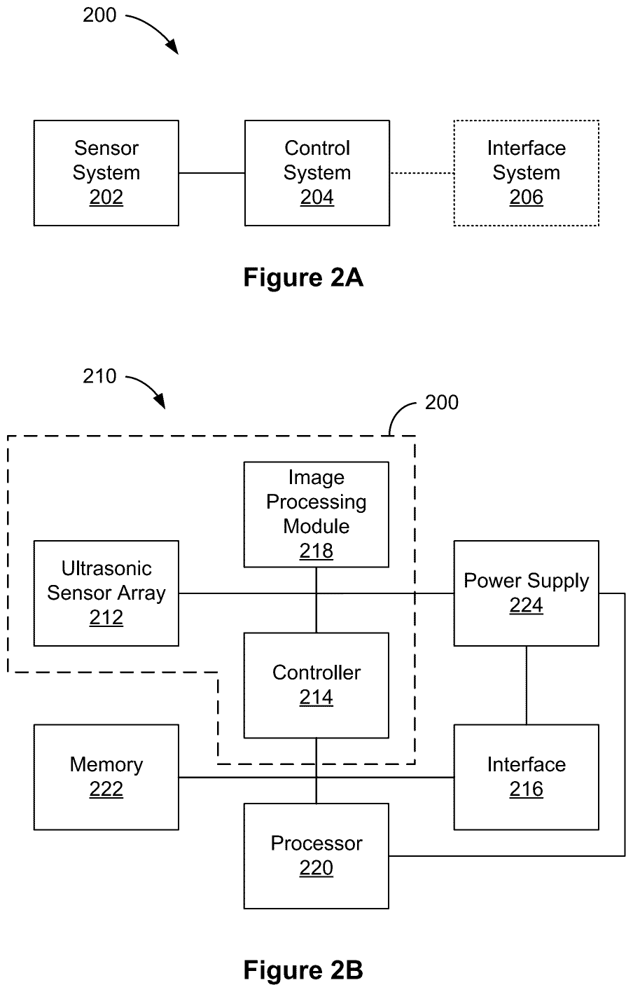

An ultrasonic sensor array may be part of a sensing system of a device, for example, apparatus 100 implemented with a mobile device. FIG. 2A shows a block diagram representation of components of an example sensing system 200. As shown, the sensing system 200 may include a sensor system 202 and a control system 204 that may, in some implementations, be electrically and/or communicatively coupled to the sensor system 202. In some implementations, control system 204 may include one or more controllers or processors. Control system 204 may be an example of control system 106. In some configurations, the control system 204 may be part of the device having the sensing system. In some configurations, the control system 204 may be part of the sensing system. In some configurations, the control system 204 may be external to the device having the sensing system, for example but not limited to, on a server (cloud), remote storage, or another device other than the device having the sensing system. In some configurations, the one or more controllers or processors may be distributed across two or more devices including external apparatus.

In some examples, the sensor system 202 may include at least the acoustic sensing system 104. In some examples, the sensor system 202 may include at least the flexible substrate 103 and the acoustic sensing system 104. The sensor system 202 (e.g., in conjunction with control system 204, in some implementations) may be capable of detecting the presence of an object, for example a human finger. The sensor system 202 may be capable of scanning an object and providing raw measured image information usable to obtain an object signature, for example, a fingerprint of a human finger (such as 350). The control system 204 may be capable of controlling the sensor system 202 and processing the raw measured image information received from the sensor system. In some implementations, the sensing system 200 may include an interface system 206 capable of transmitting or receiving data, such as raw or processed measured image information, to or from various components within or integrated with the sensing system 200 or, in some implementations, to or from various components, devices or other systems external to the sensing system.

FIG. 2B shows a block diagram representation of components of an example mobile device 210 that includes the sensing system 200 of FIG. 2A. The sensor system 202 of the sensing system 200 of the mobile device 210 may be implemented with an ultrasonic sensor array 212, such as the ultrasonic sensor array 300 shown in FIG. 3B. The control system 204 of the sensing system 200 may be implemented with a controller 214 that is electrically coupled to the ultrasonic sensor array 212. While the controller 214 is shown and described as a single component, in some implementations, the controller 214 may collectively refer to two or more distinct control units or processing units in electrical communication with one another. In some implementations, the controller 214 may include one or more of a general purpose single- or multi-chip processor, a central processing unit (CPU), a digital signal processor (DSP), an applications processor, an application specific integrated circuit (ASIC), a field programmable gate array (FPGA) or other programmable logic device (PLD), discrete gate or transistor logic, discrete hardware components, or any combination thereof designed to perform the functions and operations described herein.

The sensing system 200 of FIG. 2B may include an image processing module 218. In some implementations, raw measured image information provided by the ultrasonic sensor array 212 may be sent, transmitted, communicated or otherwise provided to the image processing module 218. The image processing module 218 may include any suitable combination of hardware, firmware and software configured, adapted or otherwise operable to process the image information provided by the ultrasonic sensor array 212. In some implementations, the image processing module 218 may include signal or image processing circuits or circuit components including, for example, amplifiers (such as instrumentation amplifiers or buffer amplifiers), analog or digital mixers or multipliers, switches, analog-to-digital converters (ADCs), passive or active analog filters, among others. In some implementations, one or more of such circuits or circuit components may be integrated within the controller 214, for example, where the controller 214 is implemented as a system-on-chip (SoC) or a system-in-package (SIP). In some implementations, one or more of such circuits or circuit components may be integrated within a DSP included within or coupled to the controller 214. In some implementations, the image processing module 218 may be implemented at least partially via software. For example, one or more functions of, or operations performed by, one or more of the circuits or circuit components just described may instead be performed by one or more software modules executing, for example, in a processing unit of the controller 214 (such as in a general purpose processor or a DSP).

In some implementations, in addition to the sensing system 200, the mobile device 210 may include a separate processor 220 such as an applications processor, a memory 222, an interface 216 and a power supply 224. In some implementations, the controller 214 of the sensing system 200 may control the ultrasonic sensor array 212 and the image processing module 218, and the processor 220 of the mobile device 210 may control other components of the mobile device 210. In some implementations, the processor 220 may communicate data to the controller 214 including, for example, instructions or commands. In some such implementations, the controller 214 may communicate data to the processor 220 including, for example, raw or processed image information. It should also be understood that, in some other implementations, the functionality of the controller 214 may be implemented entirely, or at least partially, by the processor 220. In some such implementations, a separate controller 214 for the sensing system 200 may not be required because the functions of the controller 214 may be performed by the processor 220 of the mobile device 210.

Depending on the implementation, one or both of the controller 214 and processor 220 may store data in the memory 222. For example, the data stored in the memory 222 may include raw measured image information, filtered or otherwise processed image information, estimated PSF or estimated image information, and final refined PSF or final refined image information. The memory 222 may store processor-executable code or other executable computer-readable instructions capable of execution by one or both of the controller 214 and the processor 220 to perform various operations (or to cause other components such as the ultrasonic sensor array 212, the image processing module 218, or other modules to perform operations), including any of the calculations, computations, estimations or other determinations described herein (including those presented in any of the equations below). It should also be understood that the memory 222 may collectively refer to one or more memory devices (or “components”). For example, depending on the implementation, the controller 214 may have access to and store data in a different memory device than the processor 220. In some implementations, one or more of the memory components may be implemented as a NOR- or NAND-based Flash memory array. In some other implementations, one or more of the memory components may be implemented as a different type of non-volatile memory. Additionally, in some implementations, one or more of the memory components may include a volatile memory array such as, for example, a type of RAM.

In some implementations, the controller 214 or the processor 220 may communicate data stored in the memory 222 or data received directly from the image processing module 218 through an interface 216. For example, such communicated data can include image information or data derived or otherwise determined from image information. The interface 216 may collectively refer to one or more interfaces of one or more various types. In some implementations, the interface 216 may include a memory interface for receiving data from or storing data to an external memory such as a removable memory device. Additionally or alternatively, the interface 216 may include one or more wireless network interfaces or one or more wired network interfaces enabling the transfer of raw or processed data to, as well as the reception of data from, an external computing device, system or server.

A power supply 224 may provide power to some or all of the components in the mobile device 210. The power supply 224 may include one or more of a variety of energy storage devices. For example, the power supply 224 may include a rechargeable battery, such as a nickel-cadmium battery or a lithium-ion battery. Additionally or alternatively, the power supply 224 may include one or more supercapacitors. In some implementations, the power supply 224 may be chargeable (or “rechargeable”) using power accessed from, for example, a wall socket (or “outlet”) or a photovoltaic device (or “solar cell” or “solar cell array”) integrated with the mobile device 210. Additionally or alternatively, the power supply 224 may be wirelessly chargeable.

As used herein, the term “processing unit” refers to any combination of one or more of a controller of an ultrasonic system (for example, the controller 214), an image processing module (for example, the image processing module 218), or a separate processor of a device that includes the ultrasonic system (for example, the processor 220). In other words, operations that are described below as being performed by or using a processing unit may be performed by one or more of a controller of the ultrasonic system, an image processing module, or a separate processor of a device that includes the sensing system.

FIG. 3A illustrates a side view of an example configuration of an ultrasonic sensor array of sensor elements which is capable of ultrasonic imaging. FIG. 3A depicts an ultrasonic sensor array 300 with an array of sensor elements configured as transmitting and receiving elements that may be used for ultrasonic imaging. In some implementations, the ultrasonic sensor array 300 may be an example of or a portion of a sensor element or a sensor as discussed herein.

Sensor elements 362 on a sensor array substrate 360 may emit and detect ultrasonic waves. In some implementations, sensor array substrate 360 may be an example of the flexible substrate 103 discussed above, and may thus be flexible (e.g., foldable). As illustrated, an ultrasonic wave 364 may be transmitted from at one or more sensor elements 362. The ultrasonic wave 364 may travel through a propagation medium such as an acoustic coupling medium 365 and a platen 390 towards an object 350 such as a finger or a stylus positioned on an outer surface of the platen 390. Platen 390 may be an example of platen 101, and may thus be flexible (e.g., foldable) in some implementations. A portion of the ultrasonic wave 364 may be transmitted through the platen 390 and into the object 350, while a second portion is reflected from the surface of platen 390 back towards a sensor element 362. The amplitude of the reflected wave may depend in part on the acoustic properties of the object 350 and the platen 390. The reflected wave may be detected by the sensor elements 362, from which an image of the object 350 may be acquired. For example, with sensor arrays having a pitch of about 50 microns (about 500 pixels per inch), ridges and valleys of a fingerprint may be detected. An acoustic coupling medium 365, such as an adhesive, gel, a compliant layer or other acoustic coupling material may be provided to improve coupling between an array of sensor elements 362 disposed on the sensor array substrate 360 and the platen 390. The acoustic coupling medium 365 may aid in the transmission of ultrasonic waves to and from the sensor elements 362. The platen 390 may include, for example, a layer of glass, plastic, sapphire, metal, metal alloy, or other platen material. An acoustic impedance matching layer (not shown) may be disposed on an outer surface of the platen 390. The platen 390 may include a coating (not shown) on the outer surface. In some implementations, sensor elements may be co-fabricated with thin-film transistor (TFT) circuitry or CMOS circuitry on or in the same substrate, which may be a silicon, silicon on insulator (SOI), glass or plastic substrate, in some examples. The TFT, silicon or semiconductor substrate may include row and column addressing electronics, multiplexers, local amplification stages and control circuitry.

FIG. 3B shows an example configuration of an ultrasonic sensor array including sensor elements 302 and sensor elements 304 formed on a substrate 360. Substrate 360 may be an example of the sensor array substrate 360 mentioned above. The sensor elements 302 are shown as circular sensor elements. In some implementations, the sensor elements 302 are not used for force detection in the non-ultrasonic force detection mode. Sensor elements 304 are larger than the sensor elements 302 and are shown as rectangular. It will be understood that these sensor elements 302, 304 may be any appropriate shape and size. In some implementations, the sensor elements 304 that are used for non-ultrasonic force detection may be larger than the sensor elements 302 that are used solely for ultrasonic imaging. The sensor elements 304, used during non-ultrasonic force detection mode to detect applied force as described above, are located on the periphery of the ultrasonic sensor array 300. By placing the sensor elements 304 used for force detection around the periphery, the ultrasonic sensor array may be used for centering detection. While only the sensor elements 304 are used for non-ultrasonic force detection, both sensor elements 302 and sensor elements 304 may be used for ultrasonic imaging as described above with respect to FIG. 3A. That is, the sensor elements 304 may initially be used to statically detect force from a finger press and then be switched to an ultrasonic mode for ultrasonic imaging in some implementations. In alternative implementations, the sensor elements 304 may be used only for force detection, with only the sensor elements 302 used for ultrasonic imaging. In some implementations, sensor elements 304 near the periphery of the ultrasonic sensor array 300 may be used for cursor, pointer or icon control, or for screen navigation on a display of a mobile device. In some implementations, some or all of sensor elements 302, 304, 362 in FIGS. 3A and 3B may be piezoelectric micromachined ultrasonic transducers (PMUT) and/or capacitive micromachined ultrasonic transducers (CMUT) sensor elements.

### Example Sensor Stacks

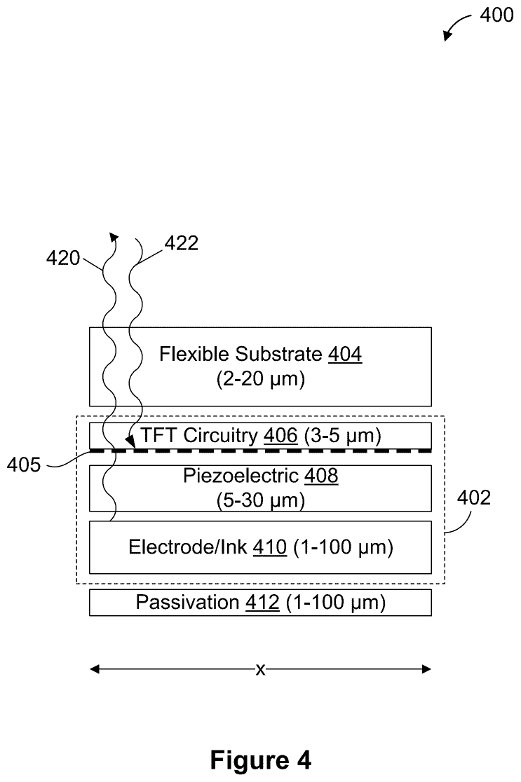

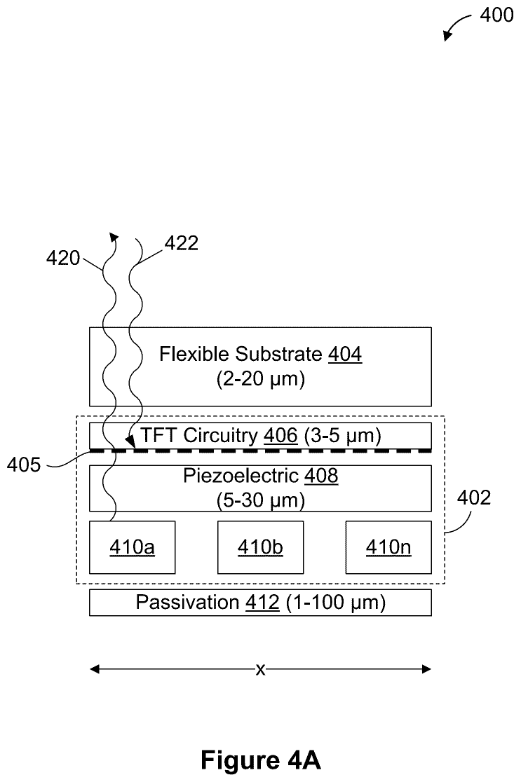

FIG. 4 is a cross-sectional diagram of an example stack of materials 400 usable with embodiments of the flexible acoustic sensor system disclosed herein. In some embodiments, the example stack of materials 400 may include a sensing element 402 and a substrate 404. In some implementations, the sensing element 402 may include TFT circuitry 406, a piezoelectric layer 408, an electrode layer 410, and a passivation layer 412.

The example stacks illustrated in the Figures are not necessarily depicted to scale.

As noted earlier, a “sensing element” may refer collectively to a transmitter element and a receiver element, such as an acoustic (e.g., ultrasonic) transmitter element and an acoustic (e.g., ultrasonic) receiver element. In some applications, the sensing element may be a fingerprint sensor or a part thereof. Hence, in some embodiments, the sensing element 402 may include an acoustic transmitter element and an acoustic receiver element, which may be examples of acoustic transmitter system 104a and acoustic receiver system 104b.

In some embodiments, the substrate 404 may be constructed of a flexible material and thus may be a flexible substrate, which may be an example of flexible substrate 103. In some implementations, the substrate 404 may comprise polyimide, and have a thickness between about 5 to 80 μm. In some implementations, the substrate 404 may comprise another polymer, such as those listed above. As such, the sensing element 402 may conform to a curved surface (such as a curved platen, foldable display, acoustic lens, etc.). In some cases, the sensing element 402 may be directly laminated to a curved surface via the substrate 404.

In some implementations, the sensing element 402 may be a flexible acoustic sensor element that is disposed adjacent to other components such as a flexible substrate, e.g., substrate 404. In some configurations, by virtue of the flexibility possessed by the sensing element 402, at least portions of the sensing element 502, as well as the substrate 404, may deform and conform to a curved surface.

In some configurations, the substrate 404 may also include components (not shown) that form a system with the sensing element 402, such as passive components, a control system (e.g., control circuitry such as an ASIC and/or a processor apparatus having one or more processors), and/or other components. These components may be electrically and/or communicatively coupled with at least the sensing element 402, enabling signal and/or data communication between the sensing element 402 and the components. For example, a transmit signal may be sent from the control system to the sensing element 402 (e.g., to an acoustic transmitter element such as the electrode layer 410), and a receive signal from the sensing element 402 (e.g., from an acoustic receiver element such as TFT circuitry 406 and/or a receiver pixel 405) may be received at the control system.

In some embodiments, the sensing element 402 may be configured to transmit one or more acoustic signals 420 (e.g., ultrasonic waves). For example, the acoustic signals 420 may travel toward a platen (not shown) and/or a target object (e.g., a body part of a user, such as a finger placed against the platen). In some configurations, the one or more acoustic signals 420 may be generated based on the transmit signal applied to the electrode layer 410.

The sensing element 402 may be further configured to receive and detect one or more returning acoustic signals 422 (e.g., reflected ultrasonic waves) from, e.g., the target object. In some implementations, thin-film transistors (TFTs) may be grown on the flexible substrate 404 (e.g., through a fabrication process) and thereby form the TFT circuitry 406. TFT circuitry 406 may include one or more discrete (or pixelated) portions that form at least part of corresponding one or more acoustic receiver elements (represented by one or more receiver pixels 405, each of which forms part of the TFT circuitry 406), in conjunction with the piezoelectric layer 408. In some examples, the one or more pixelated portions may be one or more pixelated receiver electrodes having associated TFT circuitry of the TFT circuitry 406.

As noted elsewhere herein, one or more acoustic transmitter elements and one or more receiver elements may share and use the same piezoelectric layer 408. More specifically, in some scenarios, the one or more acoustic signals 420 may be emitted from the boundary between the piezoelectric layer 408 and the electrode layer 410, and mechanical energy from the one or more returning acoustic signals 422 received at the piezoelectric layer 408 may be converted to electrical signals that are detected by the one or more receiver pixels 405 of the TFT circuitry 406 which are disposed between the boundary between the piezoelectric layer 408 and the TFT circuitry 406. Although the layers in FIG. 4 are depicted as being separate elements, they may be in direct contact with one another with adjacent layer(s). In some cases, a layer or component may be attached (e.g., laminated via an adhesive) to another layer or component, formed on a layer, or abut against another layer.

In some examples, the layer of TFT circuitry 406 may be about 3-5 μm thick. The piezoelectric layer 408 in some implementations may include a PVDF or PVDF-TrFE copolymer. In some implementations, the piezoelectric layer 408 may include lead magnesium niobate/lead titanate (PMN-PT), lithium niobate (LiNbO₃), or a combination thereof. In some implementations, the piezoelectric layer 408 may be a multilayer piezoelectric structure, or an array of such structures. In some examples, the piezoelectric layer 408 may be about 5-30 μm thick.

The electrode layer 410 may be an example of an acoustic transmitter element or a portion thereof. In some implementations, electrode layer 410 may be spin coated or deposited, e.g., on the piezoelectric layer 408. The electrode layer 410 may be patterned or cover a larger underlying substrate area. In some implementations, the electrode layer 410 may include silver (Ag), e.g., in the form of conductive ink applied to the piezoelectric layer 408. In some implementations, the electrode layer 410 may include a thin metallic layer. In some cases, the thin metallic layer may be composed of copper (Cu), which could be pliable enough to allow the sensing element 402 conform to a curved surface. In some examples, the electrode layer 410 may be up to 100 μm thick (e.g., about 1-100 μm thick). In some examples, the electrode layer 410 may be about 5-30 μm thick. In some examples, the electrode layer 410 may be more than 30 μm thick. Frequency of the acoustic waves may depend on the chosen thickness of the electrode layer 410. In implementations in which a thicker Ag is used, Ag may be applied (e.g., printed) multiple times.

In some implementations, as shown in FIG. 4A, although the electrode layer 410 may be referred to herein as an acoustic transmitter element (or one or more acoustic transmitter elements), electrode layer 410 may include one or more electrode portions (or pixels) 410a, 410b and/or 410n, which may correspond to one or more acoustic transmitter elements (and may have a thickness of about 1-100 μm thick in different implementations). Each of electrode portions 410a, 410b, 410n may be conductive ink or layer as noted above with respect to electrode layer 410.

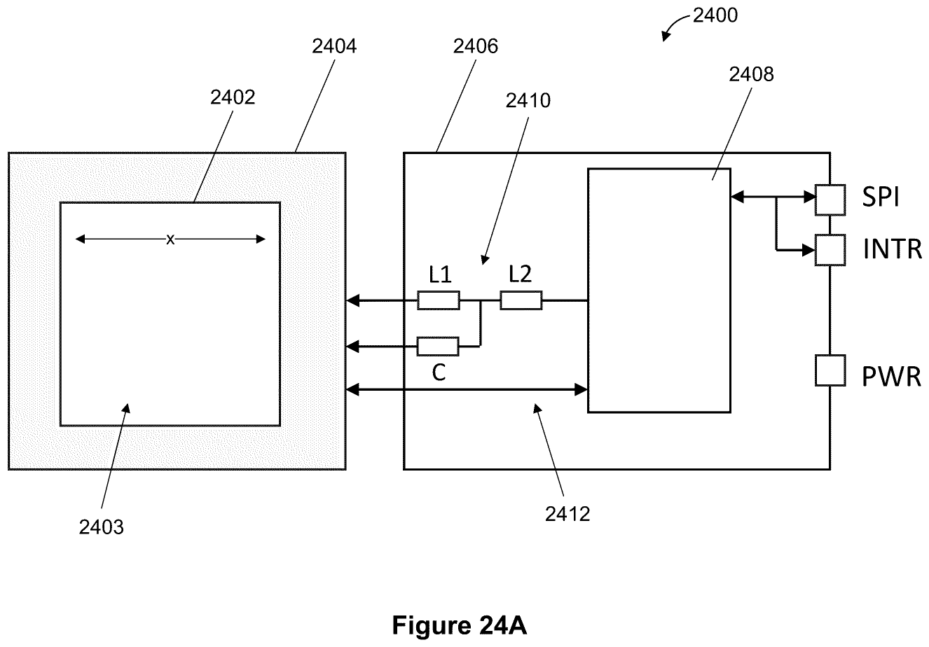

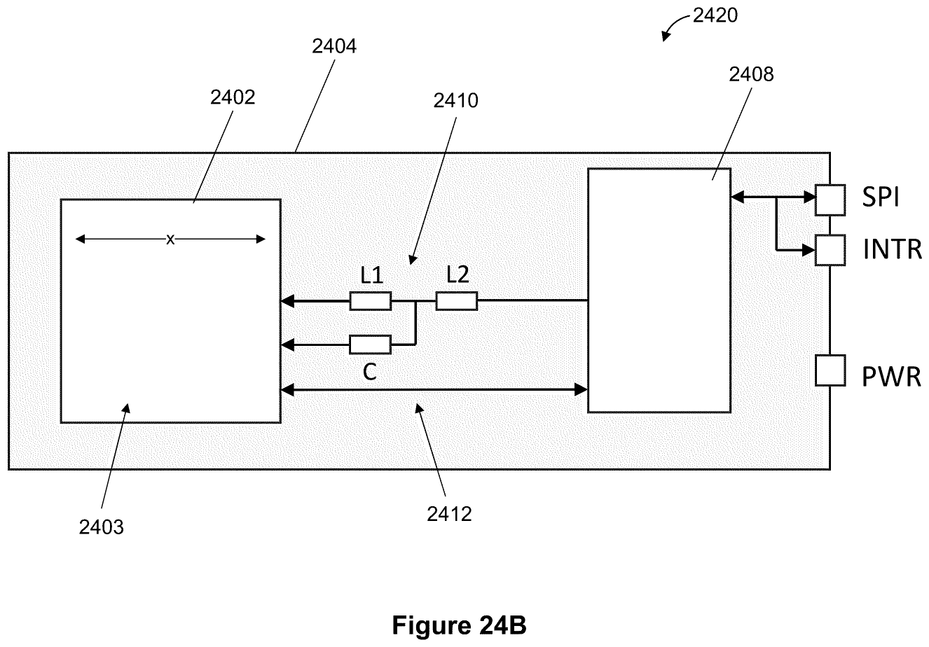

Returning back to FIG. 4, control circuitry and/or processing apparatus may drive transmit signals to the electrode layer 410, which may in turn cause generation and emission of acoustic waves from the electrode layer 410. In some examples, the control system may be configured to provide a voltage (e.g., 100-200 V, such as 120 V) to the electrode layer 410 (e.g., via a resonating circuit in passive components, further discussed with respect to FIGS. 24A and 24B), the voltage causing the electrode layer 410 to generate the one or more acoustic signals at a frequency (e.g., 1-25 MHz, such as 7, 8, 10, 12 or 15 MHz). In general, higher frequency can provide a better resolution but sacrifice on transmission (higher decibel (dB) loss). A balance may be struck when selecting the frequency. Hence, the electrode layer 410 may be configured to emit acoustic (e.g., ultrasonic) signals and function as an acoustic transmitter element.

In some implementations, a passivation layer 412 may be included with the example stack of materials 400. In some cases, passivation may 412 include a protective coating (e.g., a non-conductive ink) applied to the sensing element 402 (or a portion thereof, such as the electrode layer 410) to make the sensor element or a surface thereof less susceptible to damage (e.g., chemical reactivity, corrosion) and increase electrical stability. The ink may also affect the resonance frequency of the resonating circuit. In some cases, passivation layer 412 may include a polymer layer, such as an acrylic or other die-attached film (DAF). In some examples, the passivation layer 412 may be up to about 100 μm thick (e.g., 2-20 μm thick in some cases).

Based on the above, it can be seen that the components of the example stack of materials 400 may be made of flexible materials. More specifically, in some examples, the TFT circuitry 406 may be grown on a flexible substrate 404, the piezoelectric layer 408 may be made of copolymer, the electrode layer 410 may be made of conductive (Ag) ink or thin Cu, and passivation layer 412 may be a protective coating. Hence, the example stack of materials 400 (including the sensing element 402) may be a flexible stack and sensor element that can conform to curved surfaces and be used with flexible devices (e.g., foldable displays, wearable devices, devices with a curved surface).

Variations of the example stack of materials 400 may open avenues for use in different applications. In some cases, the example stack of materials 400 may be used with a flexible devices as noted above. In some cases, different configurations of stacks of materials having additional and/or different components as the example stack of materials 400 may result in stacks that may be used in further applications as discussed below.

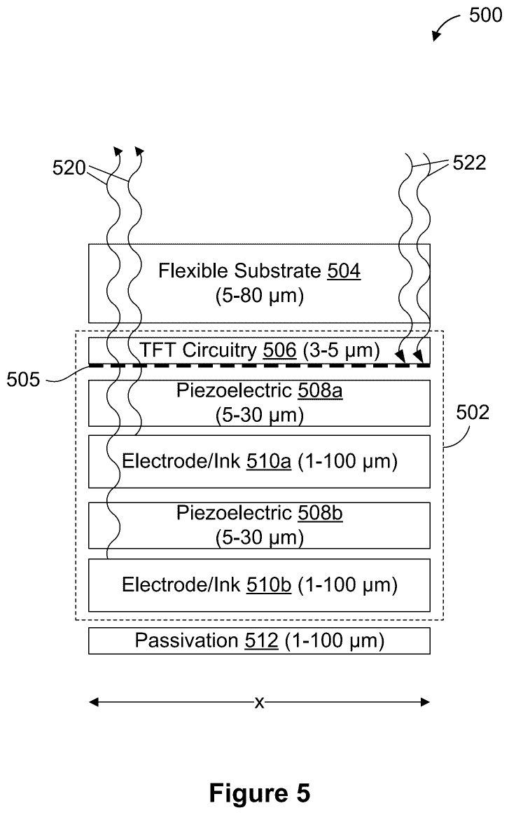

FIG. 5 is a cross-sectional diagram of another example stack of materials 500 usable with embodiments of the flexible acoustic sensor system disclosed herein. In some implementations, example stack of materials 500 may include a sensing element 502 and a substrate 504. In some implementations, the sensing element 502 may include TFT circuitry 506, at least first and second piezoelectric layers 508a and 508b, at least first and second electrode layers 510a and 510b, and a passivation layer 512. In some examples,

In some implementations, the substrate 504 may be an example of the substrate 404 and thus may be a flexible substrate constructed of a flexible material, such as polyimide. In some implementations, substrate 504 may be made of another polymer, such as those listed above. In some cases, the sensing element 502 may be directly laminated to a curved surface via the substrate 504.

In some implementations, the sensing element 502 may be disposed adjacent to other components such as a flexible substrate, e.g., the substrate 504. In some configurations, by virtue of the flexibility possessed by the sensing element 502, at least portions of the sensing element 502, as well as the substrate 504, may deform and conform to a curved surface.

Each of the piezoelectric layers 508a and 508b may be an example of piezoelectric layer 408. The passivation layer 512 may be an example of passivation layer 412.

In some embodiments, the sensing element 502 may be configured to transmit one or more acoustic signals 520 (e.g., ultrasonic waves). For example, the acoustic signals 520 may travel toward a platen (not shown) and/or a target object (e.g., a body part of a user, such as a finger placed against the platen). In some configurations, the one or more acoustic signals 520 may be generated based on the transmit signal applied to the first and/or second electrode layers 510a and/or 510b. Each of the first and second electrode layers 510a and 510b may be an example of electrode layer 410, and may be a conductive ink (e.g., Ag) or a thin metallic layer (e.g., Cu). In some cases, one of the electrode layers 510a and 510b may be a conductive ink, and the other of the electrode layers 510a and 510b a thin metallic layer. Similarly, where there are three or more electrode layers, a combination of materials may be used.

The sensing element 502 may be further configured to receive and detect one or more returning acoustic signals 522 (e.g., reflected ultrasonic waves) from, e.g., the target object. In some implementations, TFT circuitry 506 may include one or more receiver pixels 505 that form at least part of corresponding one or more acoustic receiver elements, in conjunction with the first piezoelectric layer 508a.

Control circuitry and/or processing apparatus may drive transmit signals to one or more of the first and/or second electrode layers 510a and/or 510b, which may in turn cause generation and emission of acoustic signals 520 in conjunction with first and/or second piezoelectric layers 508a and/or 508b. Similar to one or more electrode portions 410a, 410b, 410n shown in FIG. 4A, first and/or second electrode layers 510a and/or 510b may have one or more electrode portions (or pixels) corresponding to one or more acoustic transmitter elements. These pixels may be individually controlled by the control circuitry and/or processing apparatus.

In some approaches, transmissions of acoustic signals 520 and signal strength (e.g., having a higher dB) may be increased by using at least two electrode layers, e.g., 510a and 510b. As a result, more or larger (e.g., higher dB) returning acoustic signals 522 (e.g., reflected from the part of the user) may be received, which may result in additional acoustic data (e.g., fingerprint data), higher-resolution images, and greater details in the images. Moreover, the sensitivity of the receiver elements (e.g., one or more receiver pixels 505 or TFT circuitry 506) need not be high, given the quantity and larger returning acoustic signals 522. Multi-copolymer-layer sensor stacks such as example stack of materials 500 may also provide a further advantage in enabling a signal-to-noise ratio greater than single-copolymer sensor stacks (e.g., example stack of materials 400).

In addition, since portions or pixels of first and/or second electrode layers 510a and/or 510b can be individually controlled, time delays may be added to a portion of the pixels, in some implementations. By adding a time delay to certain pixels, acoustic (e.g., ultrasonic) waves may focus at certain points of convergence where there is constructive interference of the waves. This may induce a “lens effect” to the emitted acoustic signals, which may enable a stronger acoustic signal to be transmitted at points where acoustic signals constructively interfere. This approach may be advantageous in implementations where the performance of the transmitter elements is relatively lower than that of the receiver elements, or where larger acoustic signals may be desired.

Another advantage of multi-copolymer-layer sensor stacks such as the example stack of materials 500 may include “common mode cancelation.” In some configurations, a first copolymer layer (e.g., first piezoelectric layer 508a) may have a first polarization, and a second copolymer (e.g., second piezoelectric layer 508b) may have a second polarization. The first polarization direction may be the same as the second polarization, or the first polarization may be in the opposite direction of the second polarization. In implementation in which the polarization directions are opposite, the thickness of the electrode between the copolymer layers (e.g., electrode layer 510a) may be configured such that returning acoustic signals 522 at the first and second piezoelectric layers 508a and 508b may cancel the common mode. In some implementations, the thickness of the electrode layer 510a may be such that pressure of the acoustic waves are at opposite phases at the piezoelectric layers 508a and 508b.

Transmission and receipt of larger acoustic signals using example stack of materials 500 may be relevant in certain applications and use cases. Examples of these use applications may include medical devices, imaging devices, imaging probes, patches for monitoring physiological characteristics and parameters (e.g., blood flow, cardiac activities), and other medical or biometric applications. These applications may involve or benefit from usage of a flexible substrate and/or a curved surface. In some examples, sensor stacks described herein may be implemented in a wearable device, such as a patch that can be applied to the user, e.g., attached to the user's skin at the wrist or other parts of the body via a coupling film. In some cases, the wearable device may be configured to use acoustic (e.g., ultrasonic) signals to sense and/or measure biological or physiological characteristics and parameters. These example implementations will be described further below. These applications may involve obtaining information (e.g., blood flow, cardiac activities, subdermal imaging) that is more difficult to capture (e.g., compared to fingerprint sensing), or other medical or biometric applications, and may thus benefit from stronger signals and imaging resolution.

Additional layers in example stack of materials 500 may result in a thicker stack of materials, e.g., compared to example stack of materials 400. However, the form factor and chassis of devices in some aforementioned example applications may obviate the need for achieving thinnest possible sensor stacks.

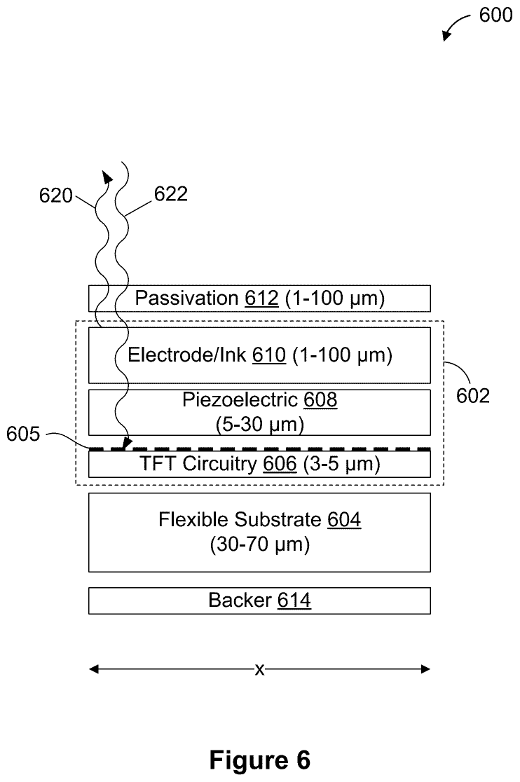

FIG. 6 is a cross-sectional diagram of another example stack of materials 600 usable with embodiments of the flexible acoustic sensor system disclosed herein. In some implementations, example stack of materials 600 may include a sensing element 602, a substrate 604, and a backing layer 614. In some implementations, the sensing element 602 may include TFT circuitry 606, a piezoelectric layer 608, an electrode layer 610, and a passivation layer 612.

In some implementations, the substrate 604 may be an example of the substrate 404 and thus may be a flexible substrate constructed of a flexible material, such as polyimide. In some implementations, substrate 604 may be made of another polymer, such as those listed above. In some cases, the sensing element 602 may be directly laminated to a curved surface via the substrate 604.

In some implementations, the sensing element 602 may be disposed adjacent to other components such as a flexible substrate, e.g., the substrate 604. In some configurations, by virtue of the flexibility possessed by the sensing element 602, at least portions of the sensing element 602, as well as the substrate 604, may deform and conform to a curved surface.

The piezoelectric layer 608 may be an example of piezoelectric layer 408. The passivation layer 612 may be an example of passivation layer 412.

In some embodiments, the sensing element 602 may be configured to transmit one or more acoustic signals 620 (e.g., ultrasonic waves). For example, the acoustic signals 620 may travel toward a platen (not shown) and/or a target object (e.g., a body part of a user, such as a finger placed against the platen). In some configurations, the one or more acoustic signals 620 may be generated based on the transmit signal applied to the electrode layer 610. The electrode layer 610 may be an example of electrode layer 410, and may be a conductive ink (e.g., Ag) or a thin metallic layer (e.g., Cu).

The sensing element 602 may be further configured to receive and detect one or more returning acoustic signals 622 (e.g., reflected ultrasonic waves) from, e.g., the target object. In some implementations, TFT circuitry 606 may include one or more receiver pixels 605 that form at least part of corresponding one or more acoustic receiver elements, in conjunction with the piezoelectric layer 608.

As can be noticed, the example stack of materials 600 may be in an orientation that is a reverse of the example stack of materials 400. That is, the piezoelectric layer 608 may be disposed between the substrate 604 and the target object, which in some examples may be a finger pressed against a display. In contrast, the substrate 404 may be disposed between the piezoelectric layer 408 and the target object (e.g., at a display). However, the sensing element 602 can generate and emit acoustic signals 620 from the electrode layer 610 and detect returning acoustic signals 622 using the one or more receiver pixels 605 of the TFT circuitry 606, as mechanical energy from the returning acoustic signals 622 are converted to electrical signals received by the TFT circuitry 606. Thus, the illustrated orientation of FIG. 6 can be used with at least similar effectiveness.

In some embodiments, the example stack of materials 600 may include the backing layer 614, disposed adjacent to the substrate 604. In some implementations, the backing layer 614 may be constructed of a polymer, such as PET, polyurethane rubber, parylene, or poly(methyl methacrylate) (PMMA). In some implementations, the backing layer 614 may be constructed of a foam or mesh, such as a porous polytetrafluoroethylene (PTFE) film (Teflon), polypropylene foam, or nylon mesh. The backing layer 614 may provide a degree of physical protection to the example stack of materials 600 and the sensing element 602, as well as improvement in performance by stabilizing or reducing forces such as stress and strain that the substrate 604 may experience, especially during bending or other deformation. Deformation can occur even without motion, such as from pressure underwater. In applications such as a wearable and/or water-resistant device (e.g., smartwatch), backing layer 614 may protect against such bending and other forces. Furthermore, it will be noted that the backing layer 614 may be used with other configurations of sensor stacks disclosed herein.

Referring to above, the range of thicknesses for the flexible substrate may be about 5-80 μm. In some implementations, the thickness of the substrate 604, which is disposed below the piezoelectric layer 608, may be between about 30 to 70 μm. In contrast, in some implementations, the thickness of the substrate 404, which is above the piezoelectric layer 408, may be between about 2 to 20 μm.

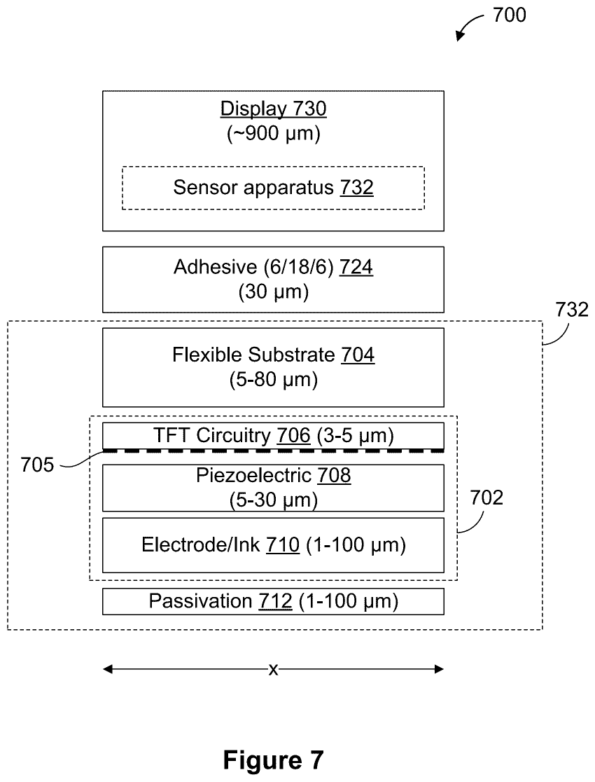

FIG. 7 is a cross-sectional diagram of an example implementation of a sensor stack 700 in a device having a display apparatus 730. In some examples, the sensor stack 700 may include the display apparatus 730, an adhesive layer 724, and a sensor apparatus 732.

In some embodiments, the sensor apparatus 732 may correspond to example stack of materials 400, and may include a substrate 704 (which may be an example of substrate 404), TFT circuitry 706 (which may be an example of TFT circuitry 406), a piezoelectric layer 708 (which may be an example of piezoelectric layer 408), an electrode layer 710 (which may be an example of electrode layer 410), and/or a passivation layer 712 (which may be an example of passivation layer 412). A sensor element 702 may comprise the TFT circuitry 706 (including one or more receiver pixels 705), piezoelectric layer 708, and electrode layer 710. In other words, the sensor apparatus 732 may correspond to or include the example stack of materials 400 (or other example stacks disclosed herein).

In some embodiments, the display apparatus 730 may include various components. Display apparatus 730 may in some cases be a flexible (e.g., foldable) display.

In some examples (e.g., in a flat-panel display), the display apparatus 730 may include a glass layer (e.g., cover glass), an optically clear adhesive (OCA) layer, a polarizing layer, one or more pressure sensitive adhesive (PSA) layers, a light-emitting layer (such as an OLED panel on a substrate, such as a polyimide or another polymer substrate), a backplate, or a combination thereof.

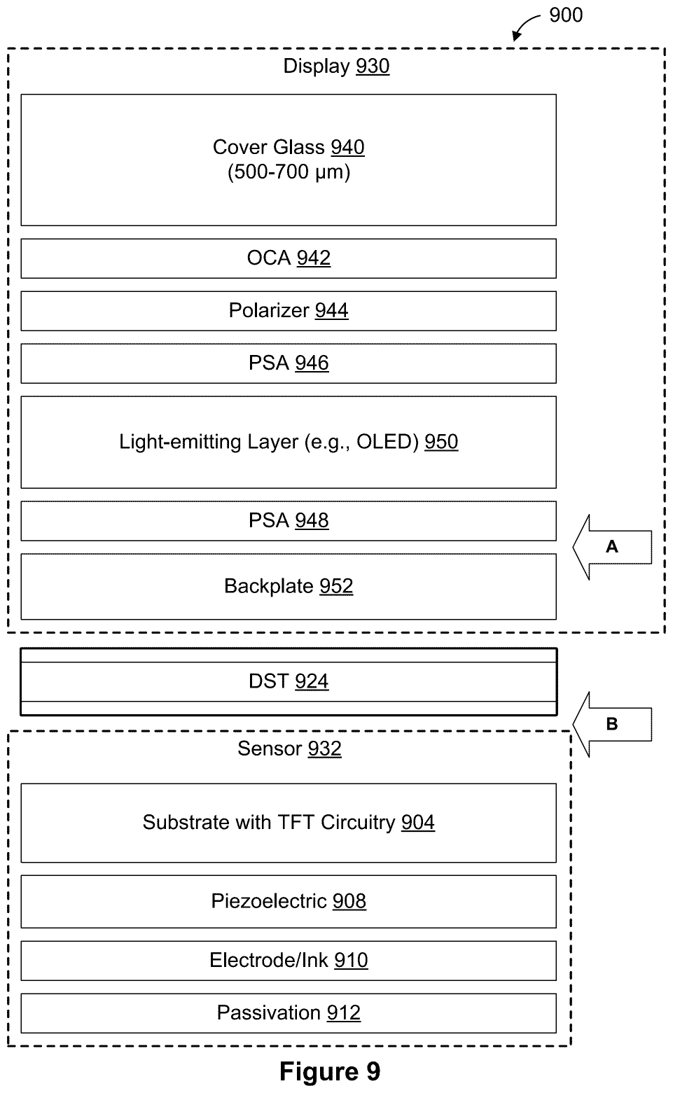

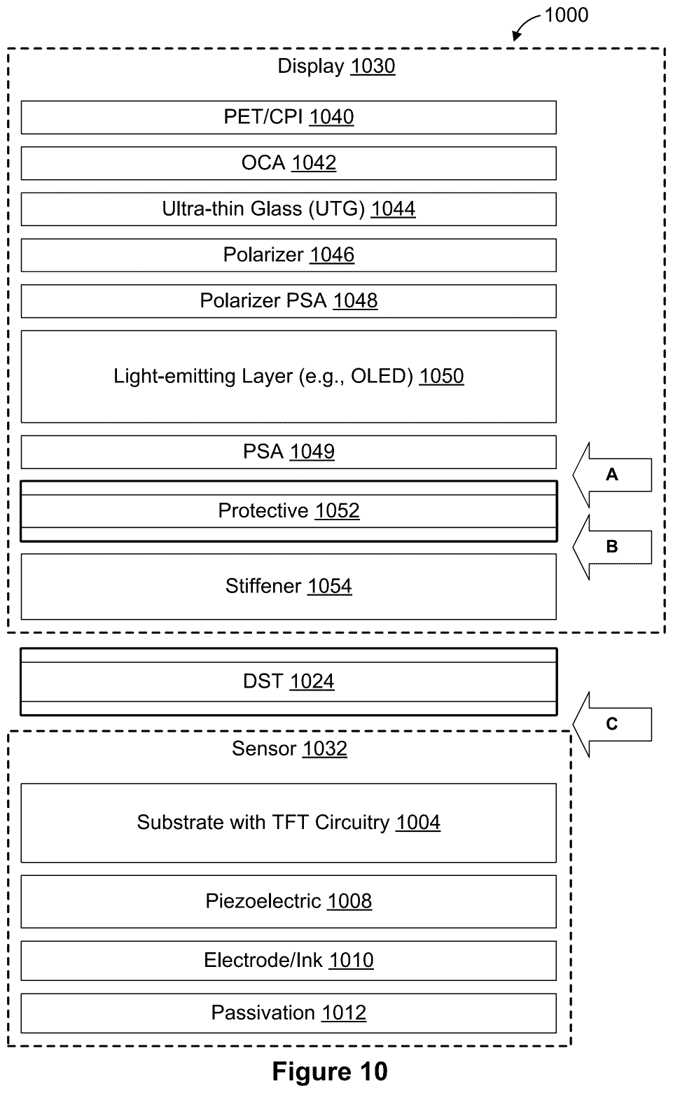

In some examples, (e.g., in a foldable display), the display apparatus 730 may include a polymer layer, an OCA layer, a glass layer (e.g., ultra-thin glass (UTG)), a polarizing layer, a light-emitting layer (which may include, e.g., an OLED panel on a substrate, such as a polyimide or other polymer substrate), one or more PSA layers, one or more protective layers (e.g., cushions, adhesives), a stiffening layer (e.g., stainless steel, titanium, aluminum, carbon fiber-reinforced polymer (CFRP)), or a combination thereof. Specific stack-up examples will be shown in FIGS. 9 and 10 and discussed below.

In some examples, the display apparatus 730 may be approximately 900 μm, although it may vary to some degree (e.g., 500-1000 μm).

In some embodiments, the sensor apparatus 732 may be secured or adhered (e.g., directly laminated) to the display apparatus 730, e.g., via an adhesive layer 724. In some implementations, the adhesive layer 724 may include a double-sided adhesive that includes a first layer of a pressure-sensitive adhesive (PSA), a layer of copper (Cu), and a second layer of PSA. In some examples, each of the PSA layers may be about 6 μm thick, and the Cu layer may be about 18 μm thick. Thus, the adhesive layer 724 may be about 30 μm thick.

In some embodiments, however, the sensor apparatus 732 may be embedded in the display apparatus 730. That is, the sensor apparatus 732 may be part of the display apparatus 730. Since the display apparatus 730 may be a flexible (e.g., foldable) display, and since the sensor apparatus 732 may also be a flexible sensor element, at least the portion of the device implementing the sensor stack 700 may be flexible (e.g., foldable).

Notably, the display apparatus 730 may be substantially thicker (e.g., about 900 μm) than the sensor apparatus 732 (e.g., about 25-135 μm, or about 100 μm in some examples). Hence, in some implementations, the display apparatus 730 may easily integrate or incorporate the sensor apparatus 732, e.g., packaged as a lightweight additional part of the display or a device, whether adjoined or laminated, or embedded therein.

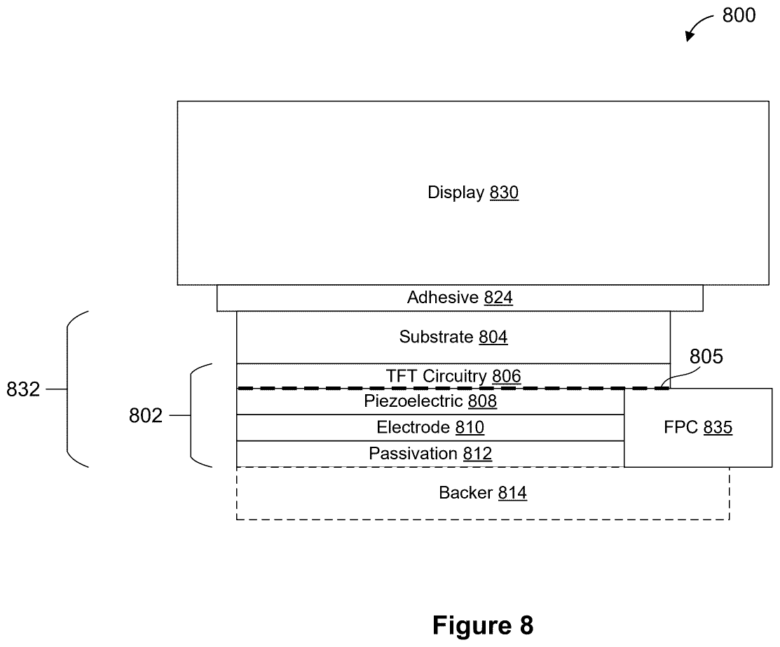

FIG. 8 is a cross-sectional diagram of another example implementation of a sensor stack 800 in a device having a display apparatus 830, according to some embodiments. In some embodiments, the sensor stack 800 may include the display apparatus 830, an adhesive layer 824, and a sensor apparatus 832.

In some embodiments, the sensor apparatus 832 may correspond to example stack of materials 400, and may include a substrate 804 (which may be an example of substrate 404), TFT circuitry 806 (which may be an example of TFT circuitry 406), a piezoelectric layer 808 (which may be an example of piezoelectric layer 408), an electrode layer 810 (which may be an example of electrode layer 410), and/or a passivation layer 812 (which may be an example of passivation layer 412). A sensor element 802 may comprise the TFT circuitry 806 (including one or more receiver pixels 805), piezoelectric layer 808, and electrode layer 810. In some implementations, the piezoelectric layer 808 may include a copolymer material coated on the one or more receiver pixels 805.

In some embodiments, the display apparatus 830 may include various components. Display apparatus 830 may in some cases be a flexible (e.g., foldable) display and may be an example of display apparatus 730.

In some embodiments, at least portions of the sensor element 802 or the sensor apparatus 832 may be attached or otherwise coupled to flexible printed circuit (FPC) 835. The FPC may function as a bridge between a control system or controlling circuitry (ASIC) and the sensor stack, e.g., to send driving signals and schemes and passing received signals to the control system. In some cases, the flexible printed circuit 835 may provide data and/or power to the sensor element 802 or the sensor apparatus 832, while retaining the physical flexibility and pliability of the sensor stack 800. For example, instructions to tune the operational frequency of the electrode layer 810 (e.g., 1-25 MHZ) may be sent to the electrode layer 810 via the flexible printed circuit 835. In some cases, the flexible printed circuit 835 may also receive and relay data from the sensor element 802 or the sensor apparatus 832 to a control system, e.g., electrical signals from received one or more returning acoustic signals.

In some embodiments, the sensor stack 800 may optionally further include a backing layer 814. In some examples, the backing layer 814 may be a foam backer including a foam material. In some implementations, the backing layer 614 may be constructed of a foam or mesh, such as a porous polytetrafluoroethylene (PTFE) film (Teflon), polypropylene foam, or nylon mesh. The backing layer 814 may provide a degree of physical protection to the stack of materials 800 and its components, as well as improvement in performance by stabilizing or reducing forces such as stress and strain that the substrate 804 may experience, especially during bending or other deformation.

FIG. 9 is a cross-sectional diagram of an example implementation of a sensor stack 900 incorporated with a display apparatus, according to some embodiments. In some embodiments, the sensor stack 900 may include at least a display apparatus 930 and a sensor apparatus 932. In some implementations, an adhesive layer 924 may also be included, which may be an example of adhesive layer 724, comprising, in some embodiments, a 6/18/6 double-sided tape (DST). The sensor apparatus 932 may be an example of sensor apparatus 732, comprising, in some embodiments, a flexible substrate 904 (e.g., polyimide) with TFT circuitry, a piezoelectric copolymer layer 908, an electrode layer 910, and/or a passivation layer 912.

The display apparatus 930 may be an example of display apparatus 730. In some embodiments, the display apparatus 930 may comprise a specific stack-up as shown, and may include a cover glass 940, an OCA layer 942, a polarizing layer 944, PSA layers 946 and 948, a light-emitting layer 950 (e.g., an OLED panel, which may be on a substrate, such as a flexible substrate, e.g., polyimide, or another polymer), and a backplate 952. In some implementations, cover glass 940 may take up about 500-700 μm in thickness. In some cases, the cover glass 940 may be an example of platen 101, which may be rigid in the sensor stack 900. The backplate 952 may be constructed of a polymer (e.g., PET) and provide physical integrity and support for the display apparatus 930. The backplate 952 need not be visually transparent.

In some configurations, the sensor apparatus 932 may be disposed adjacent to the display apparatus 930, e.g., behind the backplate 952 at position B indicated by an arrow. The sensor apparatus 932 may be directly adjoined to the display apparatus 930, laminated via the adhesive layer 924 (e.g., DST). Incidentally, the configuration of sensor stack 700 in FIG. 7 may correspond to the configuration of sensor stack 900 at position B.

In some configurations, however, the sensor apparatus 932 may be embedded in the display apparatus 930, e.g., disposed between the backplate 952 and another layer. In some examples, the sensor apparatus 932 may be disposed between one of the PSA layers (e.g., PSA layer 948) and the backplate 952 at position A indicated by an arrow. In these configurations, the sensor apparatus 932 may be placed anywhere below light-emitting layers. Also, in such configurations (e.g., where the sensor apparatus 932 is at position A or below light-emitting layer 950), the adhesive layer 924 (e.g., DST) may be omitted from the sensor stack 900, or used to attach another component. Advantageously, incorporation of the sensor apparatus 932 in the display apparatus 930 may allow for easier handling and manufacturing of the sensor stack 900 (e.g., processing steps may be omitted during fabrication of the sensor stack 900). The sensor apparatus 932 may also be packaged as a lightweight additional part of the display apparatus 930.

FIG. 10 is a cross-sectional diagram of another sensor stack 1000 incorporated with a display apparatus, according to some embodiments. In some embodiments, the sensor stack 1000 may include at least a display apparatus 1030 and a sensor apparatus 1032. In some implementations, an adhesive layer 1024 may also be included, which may be an example of adhesive layer 724, comprising, in some embodiments, a 6/18/6 double-sided tape (DST). The sensor apparatus 1032 may be an example of sensor apparatus 732, comprising, in some embodiments, a flexible substrate 1004 (e.g., polyimide) with TFT circuitry, a piezoelectric copolymer layer 1008, an electrode layer 1010, and/or a passivation layer 1012.

The display apparatus 1030 may be an example of display apparatus 730. In some embodiments, the display apparatus 1030 may comprise a specific stack-up as shown, and may be or include a flexible (e.g., foldable) display. In some embodiments, the display apparatus 1030 may include a polymer layer 1040 (e.g., polyethylene terephthalate (PET) or colorless polyimide (CPI)), an OCA layer 1042, an ultra-thin glass (UTG) layer 1044, a polarizing layer 1046, PSA layers 1048 and 1049, a light-emitting layer 1050 (e.g., an OLED panel, which may be on a substrate, such as a flexible substrate, e.g., polyimide, or another flexible polymer), one or more protective layers 1052, and a stiffening layer 1054.

In some implementations, UTG may be about 30-200 μm thick; for example, it may have a thickness of 50 μm. Its thin profile may give the glass a level of flexibility that allows it to be bent, folded, or even rolled up, which makes its usage advantageous to implementations involving flexible (e.g., foldable) devices.

In some implementations, the one or more protective layers 1052 may include adhesives (e.g., PSA, OCA and/or DST). In some implementations, the stiffening layer 1054 may include or be constructed of a metal or a polymer, e.g., stainless steel, titanium, aluminum, or CFRP. In some implementations, the stiffening layer 1054 may include or be constructed of glass. Stainless steel and glass, for example, possess high acoustic impedance. Other materials having high acoustic impedance may be used. Stiffening layer 1054 may provide physical integrity and support for the display apparatus 1030.

In some configurations, the sensor apparatus 1032 may be disposed adjacent to the display apparatus 1030, e.g., behind the stiffening layer 1054 at position C indicated by an arrow. The sensor apparatus 1032 may be directly adjoined to the display apparatus 1030, e.g., laminated via the adhesive layer 1024 (e.g., DST). Incidentally, the configuration of sensor stack 700 in FIG. 7 may correspond to the configuration of sensor stack 1000 at position C.

In some configurations, however, the sensor apparatus 1032 may be embedded in the display apparatus 1030. In some examples, the sensor apparatus 1032 may be disposed between PSA layer 1049 and the one or more protective layers 1052 at position A. In other examples, the sensor apparatus 1032 may be disposed between the one or more protective layers 1052 and the stiffening layer 1054 at position B. In these configurations, the sensor apparatus 1032 may be placed anywhere below light-emitting layers. Also, in such configurations (e.g., where the sensor apparatus 1032 is at position A or B, or below light-emitting layer 1050), the adhesive layer 1024 (e.g., DST) may be omitted from the sensor stack 1000, or used to attach another component. Advantageously, incorporation of the sensor apparatus 1032 in the display apparatus 1030 may allow for easier handling and manufacturing of the sensor stack 1000 (e.g., processing steps may be omitted during fabrication of the sensor stack 1000). The sensor apparatus 1032 may also be packaged as a lightweight additional part of the display apparatus 1030.

As such, the abovementioned materials in the sensor stack 1000 may be constructed to possess at least some flexibility and softness. The sensor apparatus 1032 may be a flexible sensor element, where its components (including, e.g., the UTG layer 1044) are constructed to conform to curved surfaces and function in flexible applications, such as foldable displays.

Advantageously, such characteristics of the components of the sensor stack 1000 may enable the display apparatus (and a device that uses the display apparatus) to be flexible (e.g., foldable) while maintaining sensor functionalities and while having a small footprint that may be appropriate for certain flexible applications. For example, fingerprint sensing may be accomplished using the acoustic sensing element in the sensor apparatus 1032, even while the device is, e.g., bent, folded, or otherwise warped into a different shape or state. Moreover, different types of sensor stacks (e.g., example stack of materials 500) may have further applicability in use cases and scenarios such as medical devices, biometric sensing, and other applications that may be more robust than, e.g., fingerprint sensing, as stated above.

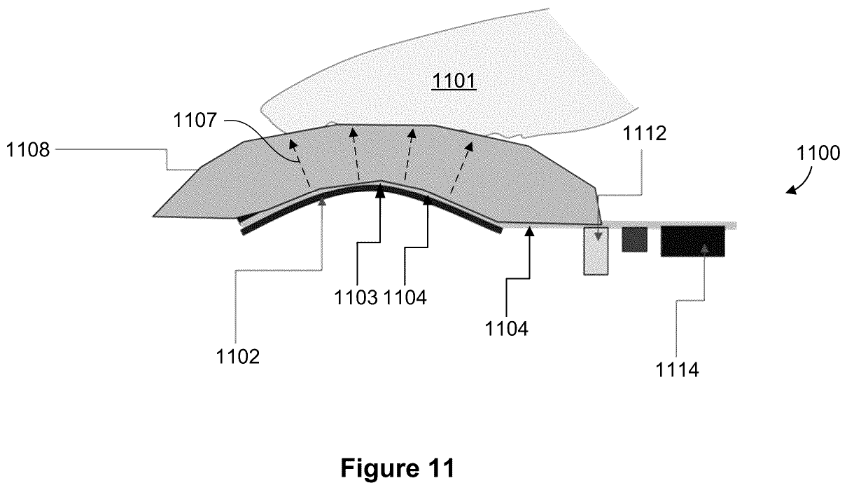

FIG. 11 is a cross-sectional diagram of a flexible sensor system 1100 using a curved surface 1108, according to some embodiments. In some embodiments, the acoustic sensor system 1100 may include a sensing element 1102 with a sensing portion associated therewith, e.g., at a surface of an acoustic transmitter element and/or an acoustic receiver element (or an array thereof) of the sensing element 1102. In some embodiments, the acoustic sensor system 1100 may further include a substrate 1104, which may be a flexible substrate and an example of substrate 404, 504, 604, 704, 804, 904 or 1004. The sensing element 1102 and the substrate 1104 may conform to a curved surface 1108, which may be part of, e.g., a platen.

In some embodiments, the curved surface 1108 may be constructed of a polymer, such as silicone rubber, polyethylene, polyethylene terephthalate (PET), polycarbonate, poly(methyl methacrylate) (PMMA). In some embodiments, the curved surface 1108 may be constructed of glass or a ceramic material. In some embodiments, the curved platen 1108 may have dimensions, curvature, angle, and other parameters that are dependent on the geometry of the device (e.g., flexible sensor system 1100) that the curved surface 1108 is implemented in. In some embodiments, the curved surface 1108 may have a curvature that causes acoustic signals 1107 traveling through the curved platen 1108 toward an object of interest (e.g., a finger 1101) to experience an altered, increased range of propagation angles. That is, the expanded propagation angle range may enable a larger imaging area associated with an imaging portion of the curved platen 1108 compared to an area associated with the sensing portion of the sensing element 1102.

In some embodiments, however, the curvature of the curved surface 1108 may result in a 1:1 imaging area with the same or substantially same area as the sensing portion of the sensing element 1102. Such 1:1 imaging may occur where the curvature of the curved surface 1108 is the same or substantially the same as the curvature of the sensing element 1102.

Nonetheless, as indicated in FIG. 11, by virtue of the curvature possessed by the curved surface 1108, acoustic signals 1107 may propagate from the sensing element 1102 at an angle relative to one another, rather than parallel to one another as they would if emitted from a planar sensing element. Reflected acoustic waves may be collected by the sensing element 1102 along the same paths as those taken by the transmitted acoustic signals 1107. That is, while the propagation angle range is expanded during transmission, the propagation angle range is narrowed when receiving the acoustic signals.

In some implementations, the curved surface 1108 may be implemented with a display. In some examples, such a display may be a curved display element 1103, such as a flexible display or a foldable display, which may be capable of bending, folding, or other distortions, or it may be fixed at, or as, a curved surface (such as the curved surface 1108). In some configurations, the curved display element 1103 may be disposed between the sensing element 1102 and the curved surface 1108, and may include components (not shown) such as a light-emitting layer (e.g., OLED), one or more adhesive layers (e.g., PSA layer and/or OCA layer), and/or a polarizing layer. In some cases, the curved surface 1108 may function as a cover surface (e.g., cover glass or other materials listed above) for the curved display element 1103, which may be disposed beneath the cover surface. In some implementations, the substrate 1104 may be a flexible substrate as noted above, and constructed to conform to a curvature of the curved platen and the curved display element.

In some configurations, the substrate 1104 may also include passive components 1112, a control system 1114 (e.g., control circuitry such as ASIC, a processor apparatus having one or more processors), and/or other components. These components may be electrically and/or communicatively coupled with at least the sensing element 1102, enabling signal and/or data communication between the sensing element 1102 and the components. For example, a transmit signal may be sent from the control system 1114 to the sensing element 1102 (e.g., to an acoustic transmitter element), and a receive signal from the sensing element 1102 (e.g., from an acoustic receiver element) may be received at the control system 1114.

An acoustic lens may not be used in some embodiments of flexible sensor system 1100. In some scenarios, the curved surface 1108 alone may allow usage of sensing element 1102 with a curved surface (including of another object of a type listed elsewhere herein). In such embodiments, the curved surface 1108 may be coupled with the sensing element 1102, e.g., via an adhesive such as adhesive layer and/or the substrate 1104 itself.

However, in other embodiments, flexible sensor system 1100 may further include an acoustic lens (not shown). Such an acoustic lens may have a curvature and/or parameters configured to alter or maintain the propagation angles and range for acoustic signals 1107. In some implementations, the curved surface 1108 may not expand the imaging area (and would result in 1:1 imaging if used alone), but it may be the acoustic lens that expands the imaging area.

In some embodiments, time delays may be added to individual pixels associated with acoustic transmitter elements. By adding a time delay to certain pixels, acoustic (e.g., ultrasonic) waves may focus at certain points of convergence where there is constructive interference of the waves. This may induce a “lens effect” to the emitted acoustic signals, which may enable a stronger acoustic signal to be transmitted at points where acoustic signals constructively interfere. This approach may be advantageous in implementations where the performance of the transmitter elements is relatively lower than that of the receiver elements.

### Example Wearable Implementations

Sensor stacks disclosed herein may be thin and flexible such that, in some embodiments, the sensor stacks may be used in flexible implementations. In some examples, as discussed above, a sensor stack may be used with a flexible device such as a foldable display. In some examples, a sensor stack may be used a wearable device such as a patch or other conformal device.

In some cases, the patch may be adhered or otherwise secured or applied to the skin of a user. The patch may be used in biomedical scenarios, such as to detect and receive acoustic signals coming from the body. In some approaches, the received signals may include ultrasonic signals that can be used to derive useful physiological characteristics, such as pulse wave velocity (PWV) associated with a target object such as an artery being measured. PWV may refer to the velocity of the pressure wave along the arterial walls. PWV is a function of the arterial wall stiffness and tension, blood density, body posture, blood pressure, and more. Other physiological characteristics derivable from ultrasonic signals may include arterial parameters, such as the size or diameter of the blood vessel over time, as well as corresponding distention or strain and heart rate waveforms. Such physiological characteristics, including PWV, may in turn be used to estimate useful physiological parameters such as blood pressure without cuffs or other tools that apply pressure to the user. Moreover, application of an external counterpressure can change the dimensions of the target blood vessel, which can complicate measurements and cause user discomfort. It would thus be valuable to obtain such information with accuracy and convenience, such as by using a flexible patch-form device having a sensor stack described herein as a biosensor that obviates the foregoing drawbacks of cuffs and other pressure-applying devices.

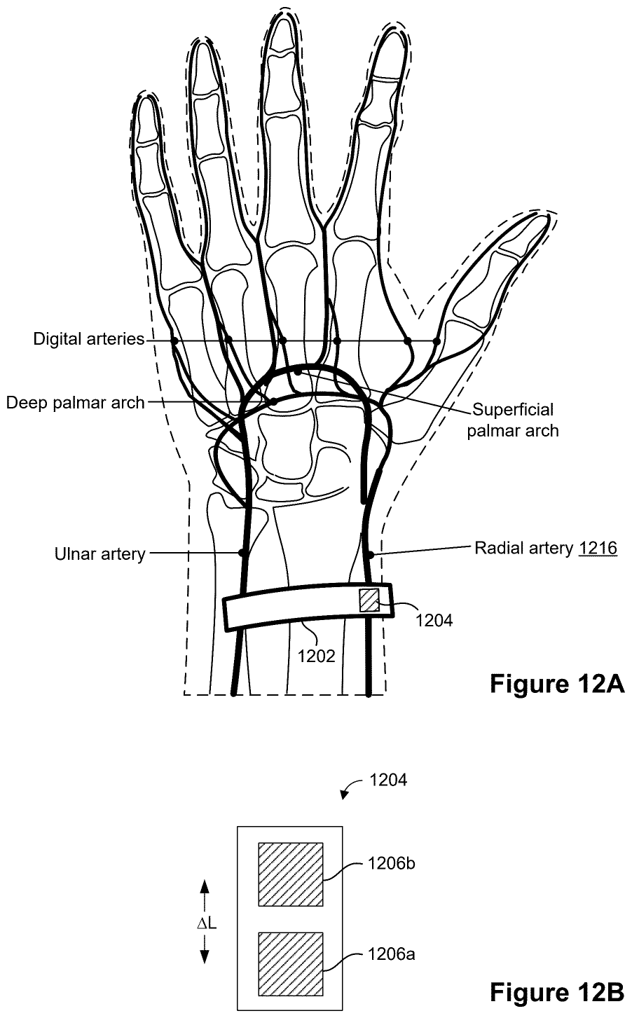

FIG. 12A illustrates an example layout of a wearable device having a sensor apparatus 1202 having a region 1204 having one or more sensor stacks of the type described herein, according to some embodiments. In some embodiments, the sensor apparatus 1202 may be part of a larger or wearable form factor, such as a wearable patch or biosensor. Hence, the sensor apparatus 1202 may be at a fixed position relative to a body part, e.g., secured or adhered to the skin while allowing acoustic signals to be exchanged with a target object at the wrist. Moreover, the region 1204 having one or more sensor stacks may be aligned such it can transmit toward and receive acoustic (e.g., ultrasonic) signals from a target object such as an artery 1216 along the body part.

FIG. 12B illustrates a simplified diagram of the region 1204 having one or more sensor stacks, shown in FIG. 12A, according to some embodiments. In some embodiments, multiple sensor stacks 1206a, 1206b may be used with the sensor apparatus 1202. Each of the sensor stacks 1206a, 1206b may be an example of the sensor stack 400, 500, 600; that is, the sensor stacks 1206a, 1206b may include a substrate having a flexible material (e.g., polyimide or other flexible polymer) that enables the sensor stacks 1206a, 1206b and ultimately sensor apparatus 1202 to conform to a non-flat surface such as a user's skin.

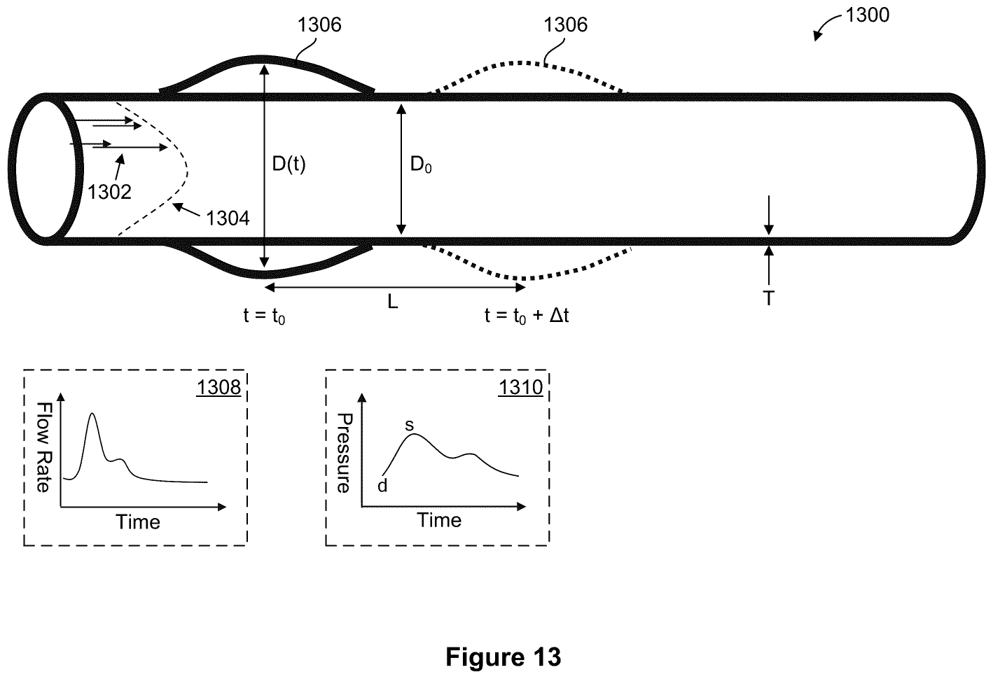

In one salient aspect, multiple sensor stacks 1206a, 1206b may allow measurement of acoustic (e.g., ultrasonic) signals to be obtained at different locations along the target object, e.g., a blood vessel such as artery 1216. Referring to FIG. 13, a cross-sectional profile of an example target object of a user during a pressure wave experienced by the example target object is illustrated. The example target object 1300 may be a blood vessel (e.g., artery 1216) in which flow of blood 1302 and its velocity profile 1304 may cause distension of the blood vessel and other changes thereto. The blood vessel may have various relevant characteristics and properties that relate to its hyper-elastic, viscoelastic, anisotropic wall. Diameter of the blood vessel at zero strain is denoted as Do. Diameter of the blood vessel during distension 1306 (e.g., maximum distension) at time to is denoted as D (t). Distension 1306 may be caused at least in part by the flood of blood 1302. Thickness of the wall of the blood vessel is denoted as T. The distension may propagate along the length of the blood vessel. For example, after time Δt has passed from time to, the distension 1306 may have traveled a length of L. The blood vessel may further be characterized by a flow rate 1308 over time and a pressure 1310 over time (including systolic(s) and diastolic (d) pressures). Other characteristics of the blood vessel may include, for example, arterial compliance, stiffness, heart rate waveform (HRW) features, and PWV. As mentioned above, PWV is the velocity of the pressure wave along the arterial wall, and is a relevant factor in determining blood pressure. An example derivation of PWV may be based on measuring photoacoustic signals at two locations separated by a distance L. Two waveforms from two locations may have a time shift t according to PWV. PWV may be estimated as L/t in this case, or ΔL/Δt generally.

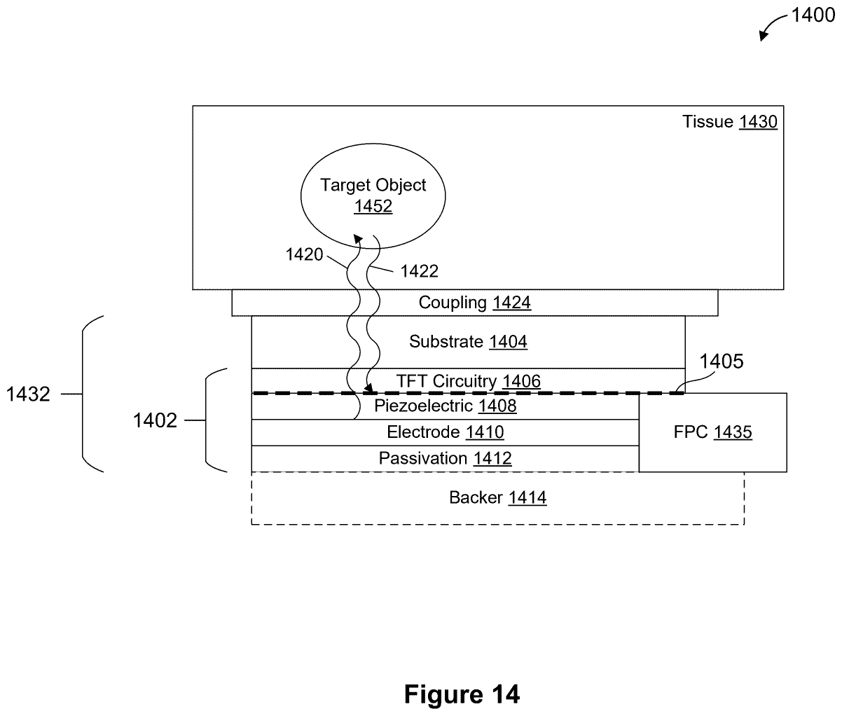

FIG. 14 is a cross-sectional diagram of another example implementation of a sensor stack 1400 in a wearable device, according to some embodiments.

In some embodiments, a sensor element 1402 may include TFT circuitry 1406 (including one or more receiver pixels 1405), a piezoelectric layer 1408, and an electrode layer 1410. In some implementations, the piezoelectric layer 1408 may include a copolymer material coated on the one or more receiver pixels 1405. Further, in some cases, the sensor element 1402 may include a passivation layer 1412.

The TFT circuitry 1406 may be an example of TFT circuitry 806 (which may be an example of TFT circuitry 406). The piezoelectric layer 1408 may be an example of piezoelectric layer 808 (which may be an example of piezoelectric layer 408). The electrode layer 1410 may be an example of electrode layer 810 (which may be an example of electrode layer 410). Thus, the sensor stack 1400 may be configured to tune an operation frequency (e.g., between 1 and 25 MHZ). The passivation layer 1412 may be an example of passivation layer 812 (which may be an example of passivation layer 412). In some embodiments, the sensor stack 1400 may optionally further include a backing layer 1414, which may be an example of backing layer 814. In some embodiments, at least portions of the sensor element 1402 or the sensor apparatus 1432 may be attached or otherwise coupled to a flexible printed circuit 1435.

In some embodiments, a sensor apparatus 1432 may correspond to example stack of materials 400, and may include a substrate 1404 and the abovementioned components of sensor element 1402. In some implementations, the substrate 1404 may be a flexible substrate having a flexible material, such as polyimide (or other flexible polymer).

In some embodiments, a coupling layer 1424 may be disposed on one side of the substrate 1404. The coupling layer 1424 may include a coupling medium (e.g., gel, adhesive such as silicone adhesive or silicone glue, or other acoustically transparent polymer having a small acoustic impedance, such as polyurethane, PMMA, or an acrylic) that can secure the sensor stack 1400 to tissue 1430. That is, the sensor stack 1400 may be implemented in a flexible and wearable device such as in a patch form and directly attached to a user's skin via the coupling layer 1424.

In some embodiments, e.g., during operation, e.g., when the sensor stack 1400 is confirmed to the skin, the sensor stack 1400 may transmit one or more acoustic signals 1420 (e.g., ultrasonic waves) from the electrode layer 1410 toward a target object 1452 such as a blood vessel, and receive one or more returning acoustic signals 1422 reflected from the target object 1452 at the one or more pixels 1405 of the TFT circuitry 1406.

In some implementations, the sensor stack 1400 may be an example of the sensor stack 1206a or 1206b. Hence, a flexible sensor apparatus such as sensor apparatus 1202 may include multiple ones of the sensor stack 1400, which would allow measurement of physiological characteristics such as PWV based on the propagation of distension of the target object 1452 using ΔL/Δt as discussed above. Other characteristics as noted above such as arterial compliance, stiffness, HRW can also be derived. In some approaches, useful physiological parameters of the user, such as blood pressure, can be estimated from PWV.

In some approaches, blood pressure may be measured at an arterial location as follows. The following version of the Bramwell-Hill equation provides a relationship of characteristics of a blood vessel, including area, pressure variation, and PWV:

$$PWV = \sqrt{\frac{A}{ρ} \frac{dP}{dA}} \qquad ( Eqn . 1 )$$

In Equation 1, p represents the density of the blood, A is the mean cross-sectional area of the blood vessel, ΔA (dA) is the difference between the maximum and minimum area of the blood vessel during a cardiac cycle, and ΔP (dP) is the difference between the central systolic and diastolic pressures. PWV is the estimated PWV obtained from the measurement of pulsatility (dA/A) and pressure variation (dP).

A modification of Equation 1 yields:

$$dP = ρ {PWV}^{2} \frac{dA}{A} \qquad ( Eqn . 2 )$$

Assuming the PWV remains relatively constant during a cardiac cycle, integrating Equation 2 can yield a pressure waveform according to Equation 3 below:

$$P ( t ) = {P}_{0} + ρ {PWV}^{2} ln ( \frac{A ( t )}{{A}_{0}} ) \qquad ( Eqn . 3 )$$

In Equation 3, P₀ represents the blood pressure at arterial area Ao.

Information required to use the above equations may be obtained using embodiments described herein. For example, ultrasound-based measurements can be used to obtain cross-sectional area of the blood vessel and dA. For example, applying, by a control system, a receiver-side beamforming process to the ultrasonic receiver signals from an array of ultrasonic receiver elements can produce a beamformed ultrasonic receiver image. In some such examples, estimating a cross-sectional area of the artery, a change in the cross-sectional area of the artery, or both, may be based at least in part on the beamformed ultrasonic receiver image. Accordingly, in some such examples, dA may be based, at least in part, on beamformed ultrasonic receiver images of an arterial cross-sectional area.

Hence, acoustic sensing implementations described above and below can be used to determine blood vessel characteristics (e.g., PWV) and physiological parameters (e.g., blood pressure). Beamforming approaches are described in further detail below.

Beamforming with Sensor Stacks

As described elsewhere above, discrete portions, also referred to as pixels, of an electrode layer or TFT circuitry may form acoustic transmitter elements and acoustic receiver elements that can each, respectively, transmit and receive acoustic (e.g., ultrasonic) signals. For example, electrode portions 410a, 410b, 410n may function as individual pixels or transmitter elements, and receiver pixels 405 may function as individual pixels or receiver elements. In some implementations, pixels may include transceiver elements each configured to perform transmitter and receiver functions, sending and receiving acoustic waves.

Such pixels have been illustrated in cross-sectional diagrams as one-dimensional rows (e.g., in FIGS. 4, 4A, 5, 6, 7, 8 and others). However, as will be recognized by those having ordinary skill in the relevant arts, the pixels may be arranged in two-dimensional (2D) arrays having rows and columns of pixels in various arrangements. For example, the pixels may be arranged such that each pixel is disposed immediately adjacent to one or more neighboring pixels in the vertical or horizontal directions (e.g., rectangular arrangement), or diagonal directions (e.g., honeycomb arrangement).

In some embodiments, a multiplexing technique such as row-column driving may be applied to these pixels. In some implementations, so-called A scans, B scans, and C scans may be employed with a 2D pixel array. A scans may refer to sampling at a single line, which may occur quickly, e.g., within microseconds (μs). B scans may be formed by performing multiple A scans, e.g., across multiple rows or multiple columns. B scans may be used to reconstruct cross-sectional images. C scans may be formed by performing scans in a two-dimensional fashion, e.g., across rows and columns, which can be used in a plane image reconstruction

Efficient scanning, beamforming, and two-dimensional and three-dimensional imaging may be performed based on the above. “Row-column driving” with C scans, or with a certain row and column, can be used to transmit and/or receive acoustic signals in a 2D pixel array. Rather than having a dedicated driver for each individual pixel (which can be complex and costly), B scans and C scans can group the pixels into rows and columns and thereby reduce the number of independent signal connections and power required compared to fully addressing each transmitter or receiver element individually. For example, N² number of interconnects for a N×N array may be reduced to 2N, and power may be needed only for selected active elements.

For instance, a selected row of acoustic transmitter elements (or pixels) may be activated to transmit an acoustic wave (e.g., ultrasound wave). For example, a 2D array of transmitter elements can be provided a transmit signal and emit ultrasonic waves toward a target object. Returning signals may then be received by a selected column of the receiver pixels. In another example, a selected column may be activated and driven with transmit signals, and receive signals may be detected along a selected row.

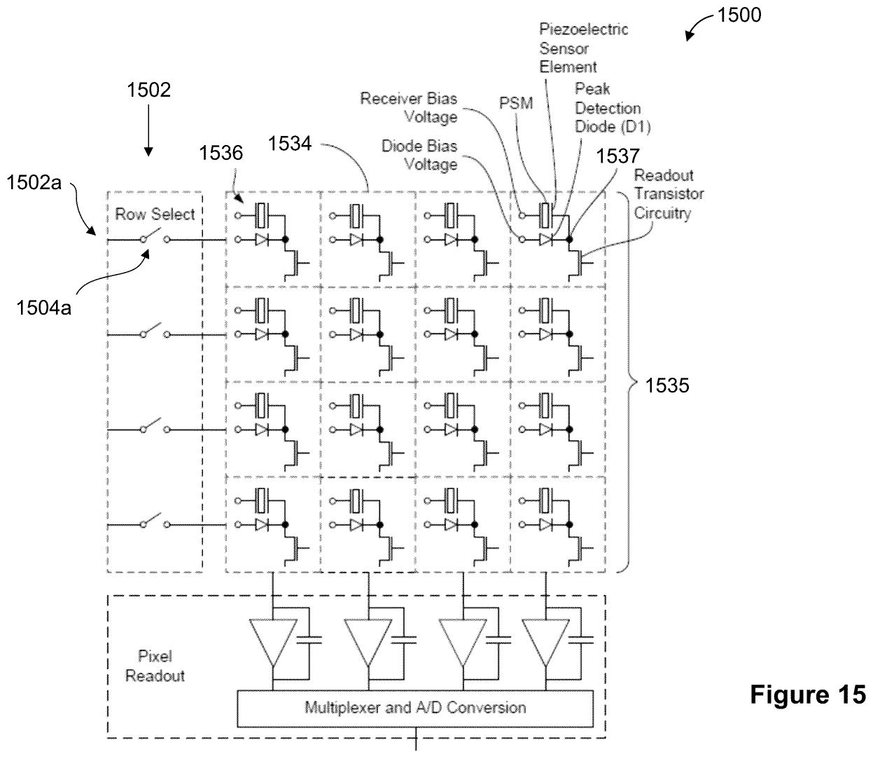

As shown in FIG. 15, an example two-dimensional array 1500 of sensor elements representationally depicts multiple rows 1502 of sensor elements capable of acoustic (e.g., ultrasonic) signaling and detection. In some examples, each sensor element (for example, sensor element 1534 of the top row) may correspond to an acoustic transmitter element, an acoustic receiver element, or an acoustic transmitter element configured to function as both an acoustic transmitter element and an acoustic receiver element. In some implementations, each sensor pixel 1534 may be, for example, associated with a local region of piezoelectric sensor material (PSM), a pixel input electrode 1537, a peak detection diode (D1) and a readout transistor circuitry (M3); many or all of these elements may be formed on or in a substrate to form a pixel circuit 1536. In practice, the local region of piezoelectric sensor material of each sensor pixel 1534 may transduce received ultrasonic energy into electrical charges. The peak detection diode D1 may register the maximum amount of charge detected by the local region of piezoelectric sensor material PSM. Each row of the pixel array 1535 may then be scanned, e.g., through a row select mechanism, a gate driver, or a shift register, and the readout transistor circuitry M3 for each column may be triggered to allow the magnitude of the peak charge for each sensor pixel 1534 to be read by additional circuitry, e.g., a multiplexer and an A/D converter. The pixel circuit 1536 may include one or more TFTs to allow gating, addressing, and resetting of the sensor pixel 1534. In some implementations, each pixel circuit 1536 may provide information about a small portion of the object detected by the ultrasonic fingerprint sensor.

In some approaches, an individual row of pixels, such as row 1502a may be activated, e.g., via a switch 1504a connecting a control system to the row 1502a of pixels. In some configurations, the activated 1502a row of pixels may be provided driving signals to emit acoustic signals at some time t₁. In some configurations, the activated 1502a row of pixels may receive and detect acoustic signals at some time t₁, while the other rows (not activated) may not detect acoustic signals. However, at subsequent times, other rows of pixels may be activated at respective times, e.g., via respective switches. For example, a second row of pixels 1502b may be activated to transmit and/or receive acoustic signals at t₂, a third row of pixels 1502c may be activated to transmit and/or receive acoustic signals at t₃, and a fourth row of pixels 1502d may be activated to transmit and/or receive acoustic signals at t₄.

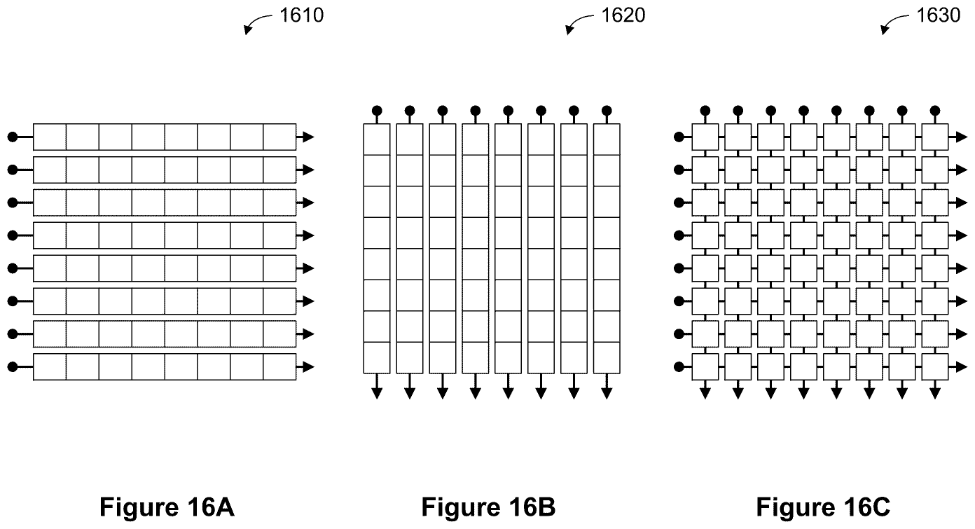

FIG. 16A illustrates an example array of sensor elements 1610 with a driving scheme applied to rows. In some configurations, a given row may be activated and provided driving signals for transmission of acoustic (e.g., ultrasonic) signals. In some configurations, a given row may be activated for receipt of acoustic (e.g., ultrasonic) signals. In some cases, rows may be activated in a sequence over time, e.g., first row at time t₁, second row at time t₂, etc. The example array of sensor elements 1610 is thus similar to that discussed with respect to FIG. 15.

FIG. 16B illustrates an example array of sensor elements 1620 with a driving scheme applied to columns. In contrast to the example array of sensor elements 1610 with a row-based driving scheme, each given column may be activated (e.g., via a switch) for transmission or receipt of acoustic signals. In some cases, columns may be activated in a sequence over time, e.g., first column at time t₁, second column at time t₂, etc.

FIG. 16C illustrates an example array of sensor elements 1630 with a driving scheme applied to both rows and columns. The example array of sensor elements 1630 may be configured to perform a combination of functionalities of the example array of sensor elements 1610 and the example array of sensor elements 1620. For example, rows may be activated in a sequence over time, e.g., first row at time tᵣ₁, second row at time tᵣ₂, etc.; subsequently, columns may be activated in a sequence over time, e.g., first row at time t_{c1}, second row at time t_{c2}, etc. That way, acoustic waves such as ultrasound waves may be transmitted by rows, and reflected ultrasound waves may be received along columns. Depth of reflectors such as artery walls traversed by emitted and reflected signals may be determined using time-of-flight calculations; e.g., depth=c*t/2, where c is the speed of sound in tissue (˜1540 m/s), and t is the time delay of the received signal.

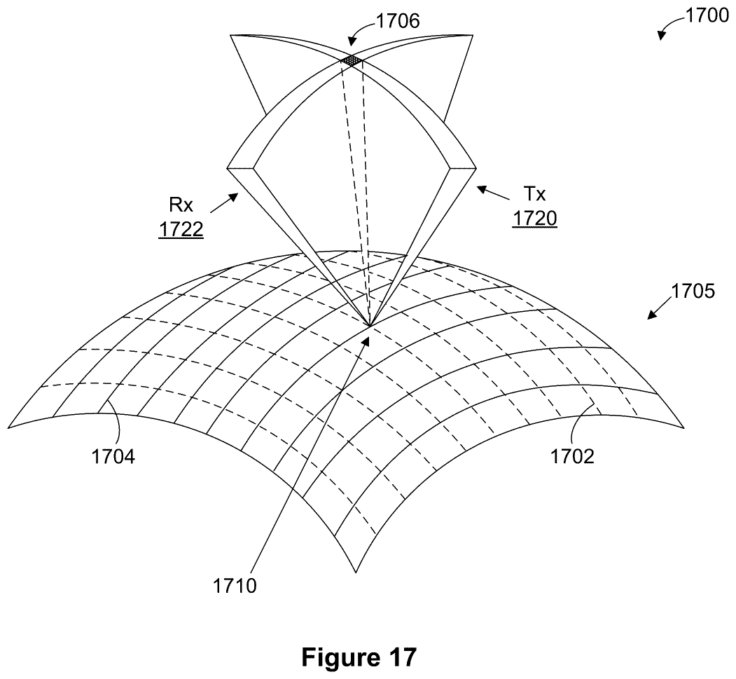

Row-column driving (e.g., as done in FIG. 16C) can perform volumetric imaging of a target object using ultrasound waves enhanced with beamforming. FIG. 17 is a simplified diagram 1700 illustrating two-way beamforming using row-column driving of a transmit beam and a receive beam with a two-dimensional sensor array 1705, according to some approaches. In some examples, the two-dimensional sensor array 1705 may include rows (or columns) of acoustic transmitter elements 1702 along dotted lines, and rows (or columns) of acoustic receiver elements 1704 along solid lines. Hence, individual rows (or columns) of acoustic transmitter elements 1702 may transmit acoustic (e.g., ultrasonic) signals 1720, and individual rows (or columns) of acoustic receiver elements 1704 may receive acoustic (e.g., ultrasonic) signals 1722.

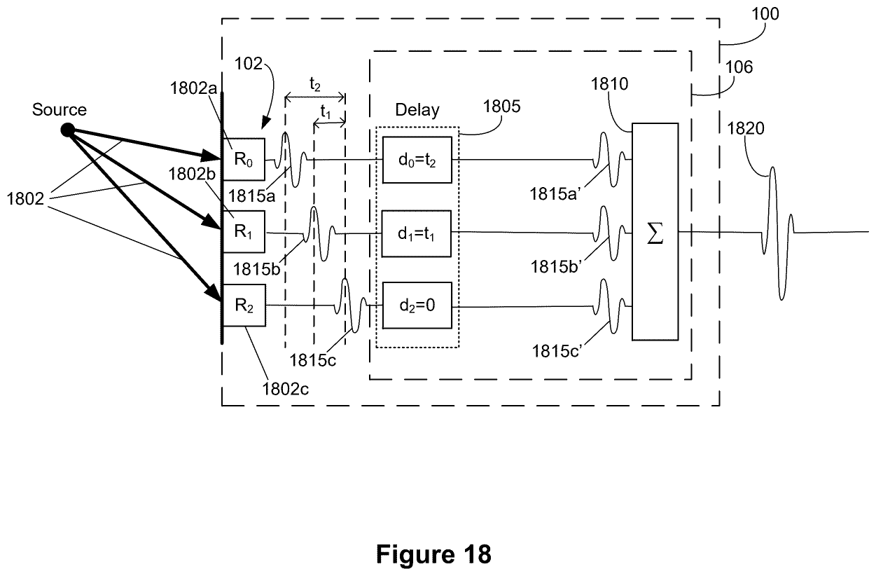

In some implementations, the two-dimensional sensor array 1705 may include flexible materials and substrate as discussed in detail above, and may be conformal to curved or irregular surfaces (e.g., skin). Even with curvature of the sensor array 1705, and extension of the array elements in the elevated direction, the transmitted and received power can be increased at the intersection 1706 of beams without sacrificing the sector shaped transmit and receive beams required for real-time volume imaging. In some approaches, a beamforming delay (such as the delay-and-sum beamforming process described with respect to FIG. 18 below) may be applied to both the rows and columns to focus the transmit and receive beams.

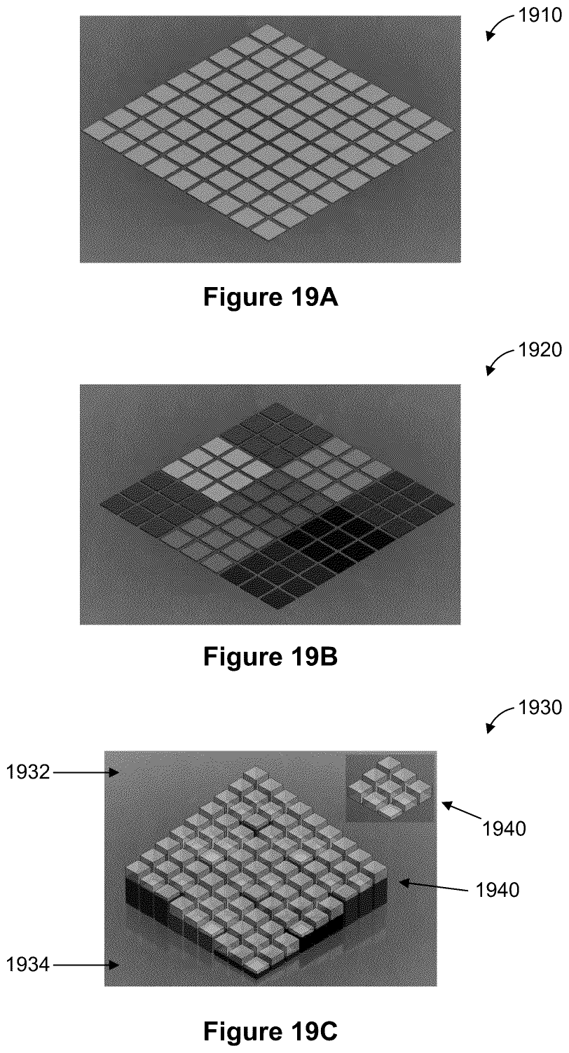

Further, in some cases, transmit and receive beams may be steered. Steering may take longer than the quick A scans. However, it may allow three-dimensional images to be obtained, as noted below after discussion of FIGS. 17 and 19C.

This type of beamforming can be useful for flexible devices such as biosensor patches. Some pixels can advantageously be elevated toward the target object, improving imaging in locations relative to the target object corresponding to where the transmit and receive beams are elevated, e.g., at intersection 1706.

In some configurations, certain sensor elements may be selected and activated for transmit and receive, e.g., where receive signals are the strongest. For instance, if determined that one or more pixels in the region 1710 of the sensor array 1705 are receiving acoustic signals having higher amplitudes, signal strengths, or power, those one or more pixels in the region 1710 sensor array 1705 may be activated for maximum beamforming performance and imaging resolution.

In some approaches, a beamforming technique such as a delay-and-sum beamforming process may be used to refine spatial resolution in combination with row-column driving. FIG. 18 shows an example of an apparatus that is configured to perform a receiver-side beamforming process. In this example, the receiver-side beamforming process is a delay-and-sum beamforming process. As with other disclosed examples, the types, numbers, sizes and arrangements of elements shown in FIG. 18 and described herein, as well as the associated described methods, are merely examples.

In this example, a source is shown emitting ultrasonic waves 1802, which are detected by active ultrasonic receiver elements 1802a, 1802b and 1802c of an array of ultrasonic receiver elements. The array of ultrasonic receiver elements may be part of a receiver system 102. The ultrasonic waves 1802 may, in some examples, correspond to the photoacoustic response of a target object to light emitted by an acoustic transmitter system 104a of the sensor apparatus 100. In this example, the active ultrasonic receiver elements 1802a, 1802b and 1802c provide ultrasonic receiver signals 1815a, 1815b and 1815c, respectively, to the control system 106.

According to this example, the control system 106 includes a delay module 1805 and a summation module 1810. In this example, the delay module 1805 is configured to determine whether a delay should be applied to each of the ultrasonic receiver signals 1815a, 1815b and 1815c, and if so, what delay will be applied. According to this example, the delay module 1805 determines that a delay do of t₂ should be applied to the ultrasonic receiver signal 1815a, that a delay d₁ of t₁ should be applied to the ultrasonic receiver signal 1815b and that no delay should be applied to the ultrasonic receiver signal 1815c. Accordingly, the delay module 1805 applies a delay of t₂ to the ultrasonic receiver signal 1815a, producing the ultrasonic receiver signal 1815a′, and applies a delay of t₁ to the ultrasonic receiver signal 1815b, producing the ultrasonic receiver signal 1815b′.

In some examples, the delay module 1805 may determine what delay, if any, to apply to an ultrasonic receiver signal by performing a correlation operation on input ultrasonic receiver signals. For example, the delay module 1805 may perform a correlation operation on the ultrasonic receiver signals 1815a and 1815c, and may determine that by applying a time shift of t₂ to the ultrasonic receiver signal 1815a, the ultrasonic receiver signal 1815a would be strongly correlated with the ultrasonic receiver signal 1815c. Similarly, the delay module 1805 may perform a correlation operation on the ultrasonic receiver signals 1815b and 1815c, and may determine that by applying a time shift of t₁ to the ultrasonic receiver signal 1815b, the ultrasonic receiver signal 1815b would be strongly correlated with the ultrasonic receiver signal 1815c.

According to this example, the summation module 1810 is configured to sum the ultrasonic receiver signals 1815a′, 1815b′ and 1815c, producing the summed signal 1820. One may observe that the amplitude of the summed signal 1820 is greater than the amplitude of any one of the ultrasonic receiver signals 1815a, 1815b or 1815c. In some instances, the signal-to-noise ratio (SNR) of the summed signal 1820 may be greater than the SNR of any of the ultrasonic receiver signals 1815a, 1815b or 1815c.

Put another way, according to this example, the control system may be configured to sum the first time-shifted ultrasonic receiver signal, the second time-shifted ultrasonic receiver signal, and the third ultrasonic receiver signal, producing a summed signal. The amplitude of the summed signal may be greater than the amplitude of any one of the first, second, or third ultrasonic receiver signal. The signal-to-noise ratio (SNR) of the summed signal may be greater than the SNR of any of the first, second, or third ultrasonic receiver signal. Hence, cleaner, stronger, and less noisy signals may be obtained by using multiple receiver elements and time-shifting certain ultrasonic signals.

Further beamforming schemes may be used with the acoustic (e.g., ultrasonic) sensor arrays described herein. FIG. 19A shows an example sensor element array 1910 of the type disclosed herein. Such a sensor element array 1910 may be a two-dimensional array having N rows of sensor elements (e.g., acoustic transmitter elements, acoustic receiver elements, acoustic transceiver elements) and N columns of sensor elements. The example sensor element array 1910 may be implemented with, or may be examples of, aforementioned embodiments, including example two-dimensional array 1500, example arrays of sensor elements 1610, 1620, 1630, and two-dimensional sensor array 1705. 2N interconnects may be used to drive transmit signals and receive signals.

Contrast example sensor element array 1910 with the example sensor element array 1920 shown in FIGS. 19B and 19C. FIG. 19B shows an example sensor element array 1920 having defined subarrays of sensor elements. In some examples, the example sensor element array 1920 may have N by N sensor elements, similar to example sensor element array 1910. However, the example sensor element array 1920 may be subdivided into subarrays, e.g., nine subarrays as shown in FIG. 19B. Interconnect complexity may be further reduced to nine (down from 2N) in this case. Further, some subarrays may correspond to particular regions of interest of a target object. In some examples, one of the subarrays may correspond to region 1710 (FIG. 17) having the strongest receive signals. There may be a coarse time delay, particularly if each subarray is used as a united sensor element, which may be refined using aforementioned beamforming technique such as a delay-and-sum to improve ultrasonic imaging resolution.

FIG. 19C shows another example sensor element array 1930 having sensor element height differentiation. At least some sensor elements may be disposed at a different height from one another. For example, sensor elements around region 1932 of the example sensor element array 1930 may be disposed at a relatively elevated height than those around region 1934. In some examples, individual sensor elements may have different heights. In some examples, sensor elements may be grouped in different domains, e.g., in a domain 1940 having nine sensor elements grouped at the same height of an underlying substrate but extended to different relative heights (e.g., using thicker layers of the sensor). In some scenarios, differing sensor element heights can compensate for different proximities of sensor elements to the target object in conformal or flexible sensor stacks. In some examples, a sensor element around region 1932 may correspond to an sensor element in region 1710 (FIG. 17) that is elevated (relative to other sensor elements) due to curvature. In this way, time delays can be made finer, and computational beamforming (such as delay-and-sum) may not be needed in at least some domains. Interconnect complexity may be further optimized in configurations such as the example sensor element array 1930 based on total number of elements, number of domains, and/or elements in a given domain. As such, time delays may be applied not based solely on signal driving scheme but by physical delays due to geometrical height.

In some implementations, at least some sensor elements may be grouped into subarrays and elevated, combining the schemes of FIGS. 19B and 19C. A given subarray may include sensor elements of the same height or different height.

In some embodiments, multiple images generated from different depths may be combined to create a three-dimensional image. Images from different depths may be obtained by, e.g., beamforming according to FIG. 17 or 19C to vary the sensor elements along the z-axis (height), or sampling receiver pixels at a depth defined by a range gate delay (RGD, discussed below) and repeating for multiple RGDs. Images from different regions about an object of interest may also be obtained by steering the beams to direct them to different regions. Additionally, real-time “four-dimensional” imaging may be performed if three-dimensional images are obtained and shown or recorded over time.

### Alternate Modalities

Embodiments disclosed herein include an acoustic sensing system, e.g., using ultrasonic waves, as illustrated in various Figures (e.g., FIGS. 1, 4, 8, 13, among others). In addition to these acoustic approaches, other types of modalities may be used to obtain measurements with respect to a target object (e.g., fingerprint, blood vessel) and images. Such other modalities may include photoacoustic, piezoelectric, and optical sensing.

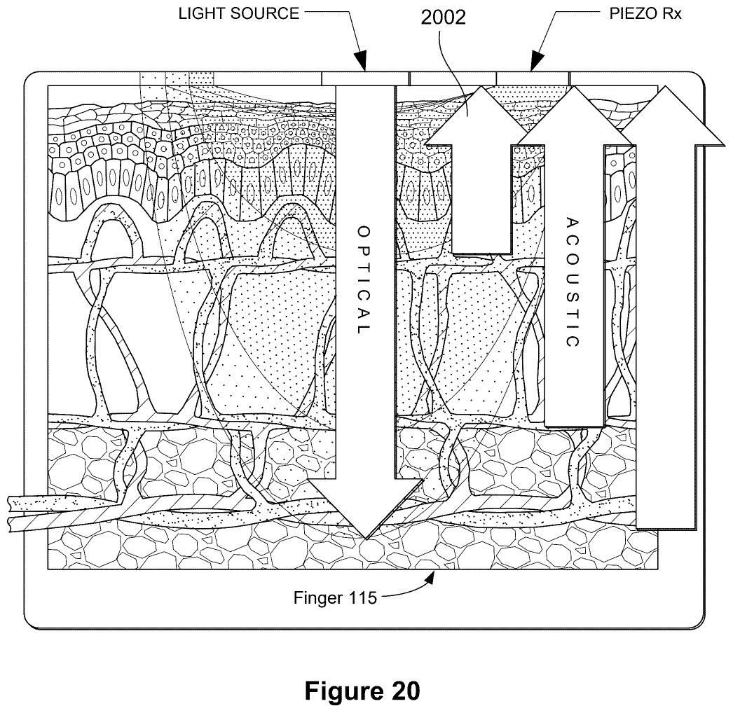

FIG. 20 shows an example of a blood pressure monitoring device based on photoacoustic plethysmography, which is referred to herein as PAPG. FIG. 20 shows the same examples of arteries, veins, arterioles, venules and capillaries inside a body part, which is a finger 115 in this example. In some examples, the light source shown in FIG. 20 may be coupled to a light source system (not shown) that is disposed remotely from the body part (e.g., finger 115). In some implementations, the light source may be an opening of an optical fiber or other waveguide. Such an opening may also be connected to an opening of an interface that is contactable with the body part. In some embodiments, the light source system may include one or more LEDs, one or more laser diodes, etc. In this example, the light source has transmitted light (in some examples, green, red, infrared, and/or near-infrared (NIR) light) that has penetrated the tissues of the finger 115 in an illuminated zone.

In the example shown in FIG. 20, blood vessels (and components of the blood itself) are heated by the incident light from the light source and are emitting acoustic waves 2002. In this example, the emitted acoustic waves 2002 include ultrasonic waves. According to this implementation, the acoustic wave emissions 2002 are being detected by an ultrasonic receiver, which is a piezoelectric receiver in this example. Photoacoustic emissions 2002 from the illuminated tissues, detected by the piezoelectric receiver, may be used to detect volumetric changes in the blood of the illuminated zone of the finger 115 that correspond to physiological data within the illuminated tissues of finger 115, such as heart rate waveforms. Although some of the tissue areas shown to be illuminated are offset from those shown to be producing photoacoustic emissions 2002, this is merely for illustrative convenience. It will be appreciated that that the illuminated tissues will actually be those producing photoacoustic emissions. Moreover, it will be appreciated that the maximum levels of photoacoustic emissions will often be produced along the same axis as the maximum levels of illumination.

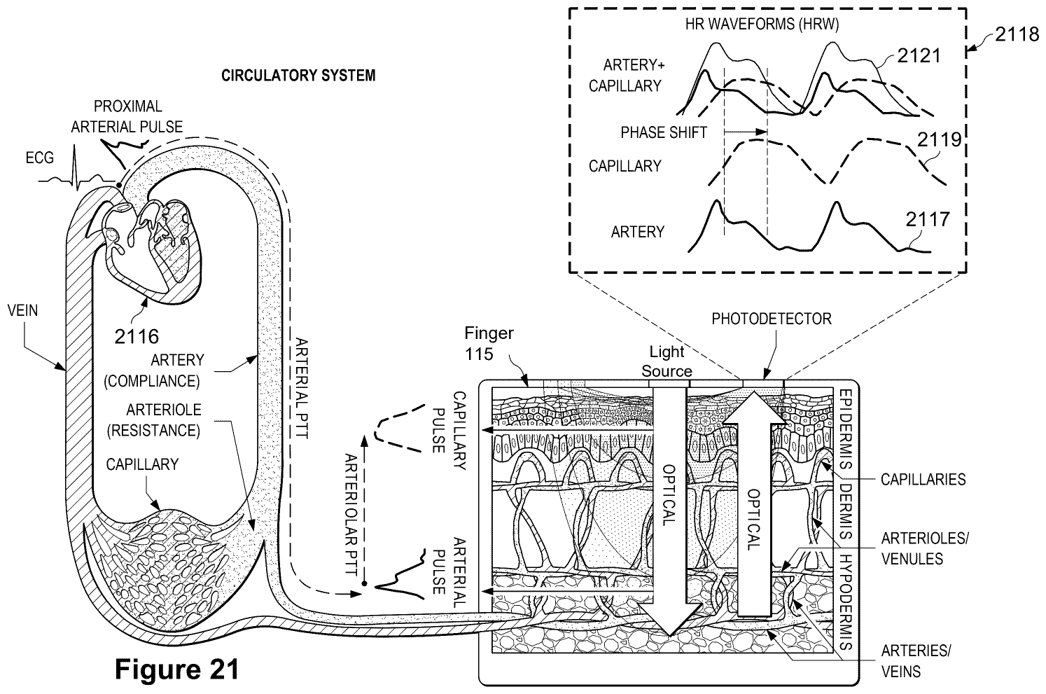

One important difference between an optical technique such as a photoplethysmography (PPG)-based system (e.g., shown in FIG. 21 below) the PAPG-based method of FIG. 20 is that the acoustic waves shown in FIG. 20 travel much more slowly than the reflected light waves involved in PPG. Accordingly, depth discrimination based on the arrival times of the acoustic waves shown in FIG. 20 is possible, whereas depth discrimination based on the arrival times of the light waves in PPG may not be possible. This depth discrimination allows some disclosed implementations to isolate acoustic waves received from the different blood vessels.

According to some such examples, such depth discrimination allows artery heart rate waveforms to be distinguished from vein heart rate waveforms and other heart rate waveforms. Therefore, blood pressure estimation based on depth-discriminated PAPG methods can be substantially more accurate than blood pressure estimation based on PPG-based methods.

FIG. 21 shows an example of a blood pressure monitoring device based on photoplethysmography (PPG). FIG. 21 shows examples of arteries, veins, arterioles, venules and capillaries of a circulatory system, including those inside a finger 115. In the example shown in FIG. 21, an electrocardiogram (ECG) sensor has detected a proximal arterial pulse near the heart 2116. Some examples are described below of measurement of the arterial pulse transit time (PTT) according to arterial pulses measured by two sensors, one of which may be an electrocardiogram sensor in some implementations.

According to the example shown in FIG. 21, a light source that includes one or more lasers or light-emitting diodes (LEDs) has transmitted light (in some examples, green, red, infrared, and/or near-infrared (NIR) light) that has penetrated the tissues of the finger 115 in an illuminated zone. Reflections from these tissues, detected by a photodetector, may be used to detect volumetric changes in the blood of the illuminated zone of the finger 115 that correspond to heart rate waveforms.

As shown in the heart rate waveform graphs 2118 of FIG. 21, the capillary heart rate waveform 2119 is differently-shaped and phase-shifted relative to the artery heart rate waveform 2117. In this simple example, the detected heart rate waveform 2121 is a combination of the capillary heart rate waveform 2119 and the artery heart rate waveform 2117. In some instances, the responses of one or more other blood vessels may also be part of the heart rate waveform 2121 detected by a PPG-based blood pressure monitoring device.

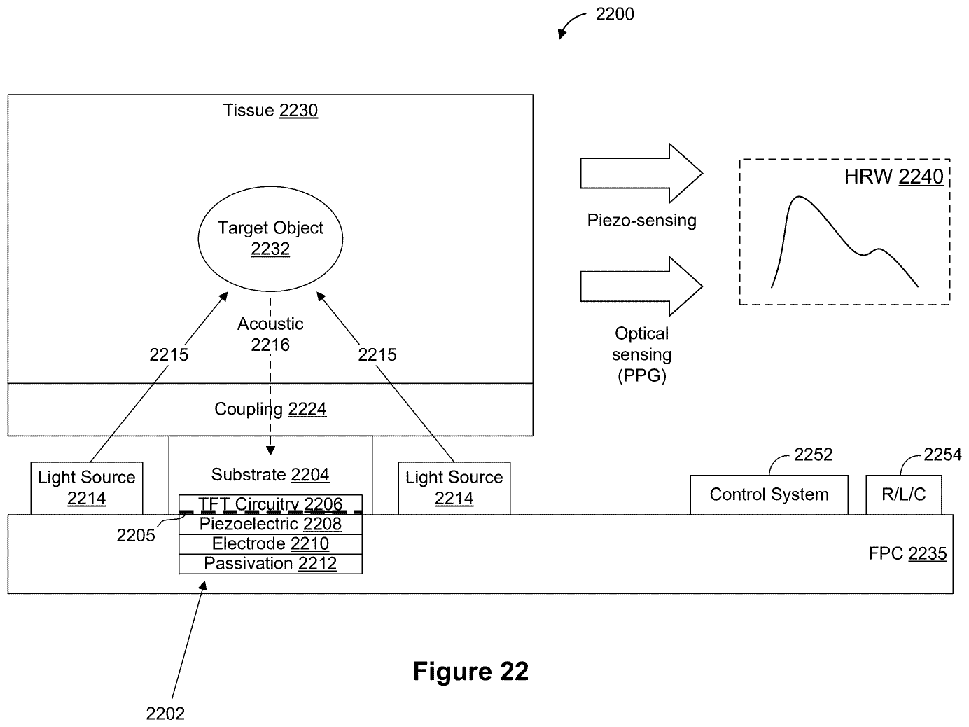

FIG. 22 shows a cross-sectional diagram of an example implementation of a sensor apparatus 2200 with photoacoustics capability, according to some embodiments. In some embodiments, the sensor apparatus 2200 may include a sensor stack 2202 having a substrate 2204, TFT circuitry 2206 (including one or more receiver pixels 2205), a piezoelectric layer 2208, and an electrode layer 2210. In some implementations, the sensor stack 2202 may further include a passivation layer 2212, and at least portions of the sensor stack 2202 may be attached or otherwise coupled to a flexible printed circuit (FPC) 2235. In some configurations, the FPC 2235 may include other components such as a control system 2252 (e.g., an ASIC and/or a processor apparatus having one or more processors) configured to communicate with one or more of the aforementioned components (e.g., receiver pixels 2205, pixels of electrode layer 2210). FPC 2235 may also include one or more passive components 2254 and other circuitry. In some embodiments, a coupling layer 2224 may be disposed on one side of the substrate 2204 as shown. The coupling layer 2224 may include a coupling medium (e.g., gel, adhesive such as silicone adhesive or silicone glue, or other acoustically transparent polymer having a small acoustic impedance, such as polyurethane, PMMA, or an acrylic) that can secure the sensor stack 2202 to tissue 2230. That is, in some implementations, the sensor stack 2202 may be implemented in a flexible and wearable device such as a patch-form biosensor and directly attached to a user's skin via the coupling layer 2224.

These components may be examples of similar ones discussed above with respect to, e.g., FIGS. 4 and 14. Hence, the substrate 2204 may be a flexible substrate composed of a flexible material such as polyimide or other flexible polymers.

In some embodiments, the sensor apparatus 2200 may include a light source system, which may include one or more light sources 2214. Each of the one or more light sources 2214 may be configured to generate and emit optical signals 2215 such as light toward a target object 2232 in the tissue 2230. Based on principles of photoacoustics as discussed above with respect to FIG. 20, an acoustic wave 2217 may be emitted from the target object 2232 that has been illuminated by optical signals 2215.

In some configurations, a light source 2214 may be in a portion of a wearable device. In some cases, the light source may be remote (e.g., on a different location on the FPC 2235), and an optical waveguide (e.g., optical fiber) may be coupled to the remote light source. In such cases, light or other optical signals may travel via the optical waveguide toward the target object 2232 (e.g., artery or other blood vessel) that is proximate to the sensor apparatus 2200.

In some embodiments, the light source system may, include one or more light-emitting diodes. In some implementations, the light source system may include one or more laser diodes. According to some implementations, the light source system may include one or more vertical-cavity surface-emitting lasers (VCSELs) and/or one or more edge-emitting lasers (EELs). In some implementations, the light source system may include one or more edge-emitting lasers. In some implementations, the light source system may include one or more neodymium-doped yttrium aluminum garnet (Nd:YAG) lasers.

Hence, the light source 2214 may be, for example, a laser diode, a light-emitting diode (LED), or an array of either or both. The light source 2214 may be configured to generate and emit optical signals 2215. The light source system may, in some examples, be configured to transmit light in one or more wavelength ranges. In some examples, the light source system may be configured to transmit light in a wavelength range of 500 to 600 nanometers (nm). According to some examples, the light source system may be configured to transmit light in a wavelength range of 800 to 950 nm. According to some examples, the light source system may be configured to transmit light in infrared or near infrared (NIR) region of the electromagnetic spectrum (about 700 to 2500 nm). In view of factors such as skin reflectance, fluence, the absorption coefficients of blood and various tissues, and skin safety limits, one or both of these wavelength ranges may be suitable for various use cases. For example, the wavelength ranges of 500 nm to 600 nm and of 800 to 950 nm may both be suitable for obtaining photoacoustic responses from relatively smaller, shallower blood vessels, such as blood vessels having diameters of approximately 0.5 mm and depths in the range of 0.5 mm to 1.5 mm, such as may be found in a finger. The wavelength range of 800 to 950 nm, or about 700 to 900 nm, or about 600 to 1100 nm may, for example, be suitable for obtaining photoacoustic responses from relatively larger, deeper blood vessels, such as blood vessels having diameters of approximately 2.0 mm and depths in the range of 2 mm to 3 mm, such as may be found in an adult wrist. In some implementations, the light source system or light source 2214 may be configured to switch wavelengths to capture acoustic information from different depths, e.g., based on signal(s) from a control system 2252 (or 106).

In some implementations, the light source system may be configured for emitting various wavelengths of light, which may be selectable to trigger acoustic wave emissions primarily from a particular type of material. That is, light sources may correspond to visible light, infrared light, or both. For example, because the hemoglobin in blood absorbs near-infrared light very strongly, in some implementations the light source system may be configured for emitting one or more wavelengths of light in the near-infrared range, in order to trigger acoustic wave emissions from hemoglobin. However, in some examples, the control system 2252 (or 106) may control the wavelength(s) of light emitted by the light source system to preferentially induce acoustic waves in blood vessels, other soft tissue, and/or bones. For example, an infrared (IR) light-emitting diode LED may be selected and a short pulse of IR light emitted to illuminate a portion of a target object and generate acoustic wave emissions that are then detected by the receiver system. In another example, an IR LED and a red LED or other color such as green, blue, white or ultraviolet (UV) may be selected and a short pulse of light emitted from each light source in turn with ultrasonic images obtained after light has been emitted from each light source. In other implementations, one or more light sources of different wavelengths may be fired in turn or simultaneously to generate acoustic emissions that may be detected by the ultrasonic receiver. Image data from the ultrasonic receiver that is obtained with light sources of different wavelengths and at different depths (e.g., varying range gate delays (RGDs)) into the target object may be combined to determine the location and type of material in the target object. Image contrast may occur as materials in the body generally absorb light at different wavelengths differently. As materials in the body absorb light at a specific wavelength, they may heat differentially and generate acoustic wave emissions with sufficiently short pulses of light having sufficient intensities. Depth contrast may be obtained with light of different wavelengths and/or intensities at each selected wavelength. That is, successive images may be obtained at a fixed RGD (which may correspond with a fixed depth into the target object) with varying light intensities and wavelengths to detect materials and their locations within a target object. For example, hemoglobin, blood glucose or blood oxygen within a blood vessel inside a target object such as a finger may be detected photoacoustically.

According to some implementations, the light source system may be configured for emitting a light pulse with a pulse width less than about 100 nanoseconds. In some implementations, the light pulse may have a pulse width between about 10 nanoseconds and about 500 nanoseconds or more. According to some examples, the light source system may be configured for emitting a plurality of light pulses at a pulse repetition frequency between 10 Hz and 100 kHz. Alternatively, or additionally, in some implementations the light source system may be configured for emitting a plurality of light pulses at a pulse repetition frequency between about 1 MHz and about 100 MHz. Alternatively, or additionally, in some implementations the light source system may be configured for emitting a plurality of light pulses at a pulse repetition frequency between about 10 Hz and about 1 MHz. In some examples, the pulse repetition frequency of the light pulses may correspond to an acoustic resonant frequency of the ultrasonic receiver and the substrate. For example, a set of four or more light pulses may be emitted from the light source system at a frequency that corresponds with the resonant frequency of a resonant acoustic cavity in the sensor stack, allowing a build-up of the received ultrasonic waves and a higher resultant signal strength. In some implementations, filtered light or light sources with specific wavelengths for detecting selected materials may be included with the light source system. In some implementations, the light source system may contain light sources such as red, green and blue LEDs of a display that may be augmented with light sources of other wavelengths (such as IR and/or UV) and with light sources of higher optical power. For example, high-power laser diodes or electronic flash units (e.g., an LED or xenon flash unit) with or without filters may be used for short-term illumination of the target object.

According to some examples, the light source system may also include one or more light-directing elements configured to direct light from the light source system towards the target object along the first axis. In some examples, the one or more light-directing elements may include at least one diffraction grating. Alternatively, or additionally, the one or more light-directing elements may include at least one lens.

In some example implementations, some or all of the one or more light sources may be disposed at or along an axis that is parallel to or angled relative to a central axis associated with the sensor apparatus 2200, e.g., relative to the FPC 2235, substrate 2204, coupling layer 2224, etc. FIG. 22 shows optical signals 2215 emitted toward the target object 2232, which may cause generation of acoustic (e.g., ultrasonic) waves 2217 by the target object 2232. These ultrasonic waves 2217 may be detectable by one or more receiver elements (e.g., receiver pixels 2205) of the sensor stack.

In various configurations, the light source system may incorporate anti-reflection (AR) coating, a mirror, a light-blocking layer, a shield to minimize crosstalk, etc.

The light source system may include various types of drive circuitry, depending on the particular implementation. In some implementations, the control system 2252 (or 106) may include the drive circuitry. In some disclosed implementations, the light source system may include at least one multi-junction laser diode, which may produce less noise than single-junction laser diodes. In some examples, the light source system may include a drive circuit (also referred to herein as drive circuitry) configured to cause the light source system to emit pulses of light at pulse widths in a range from 3 nanoseconds to 1000 nanoseconds. According to some examples, the light source system may include a drive circuit configured to cause the light source system to emit pulses of light at pulse repetition frequencies in a range from 1 kilohertz to 100 kilohertz.

Notably, in some configurations, the sensor apparatus 2200 may generate and emit both acoustic (e.g., ultrasonic) waves from the electrode layer 2210, optical signals from one or more light sources 2214 of the light source system, or a combination thereof. For example, PPG may be performed using both acoustic and ultrasonic transmitters. In some cases, the control system 2252 may cause the modality to switch between transmitting acoustic signals in one mode and optical signals in another mode.

In some embodiments, the illustrated sensor apparatus 2200 may be configured to perform passive piezo-sensing, in which the piezoelectric material (e.g., in the piezoelectric layer 2208) of the sensor apparatus may detect mechanical deformations without an external power source actively exciting the sensor or electrode 2210. As mentioned previously, the sensor apparatus 2200 and its components (such as a piezoelectric receiver layer 2208 made of PVDF copolymer) may in a wearable, flexible, conformal, and thin form factor (e.g., in a patch form) and may have direct contact with the tissue 2230 via the coupling layer 2224. As such, pressure variations detected from a target object 2232 over time, e.g., from blood flow in a blood vessel can result in, for example, distension 1306. The expansion and contraction of the blood vessel can generate and apply mechanical pressure waves to the piezoelectric layer 2208 that propagate through the blood vessel. Per piezoelectrical principles, the piezoelectric layer 2208 may generate an electric signal proportional to the applied pressure. A heart rate waveform (HRW) 2240 may be captured based on the generated electrical signal, which may provide information such as pulse wave characteristics (including, e.g., PWV), heart rate, arterial stiffness, and other physiological characteristics of the target object 2232.

In some embodiments, optical sensing may be performed according to PPG principles described above with respect to FIG. 21. In some implementations, light incident through an optically transparent substrate 2204 (e.g., polyimide) can be sensed by an exposed P-N junction of the peak detection diode (D1) of the underlying TFT circuitry 2206, e.g., at one or more receiver pixels 2205 thereof. In some configurations, the peak detection diode may be used to capture optical signals 2216.

As such, in some implementations, HRW 2240 may be captured from the optical signals. Other examples of heart rate waveform graphs 2118 that can be derived from optical signals (and, e.g., volumetric changes in the blood vessel derived based on the optical signals) are shown in FIG. 21. Physiological characteristics may be determined from the HRW 2240 and heart rate waveform graphs 2118 generated from optical sensing by the optically transparent TFT substrate.

Hence, HRW and other physiological characteristics such as PWV can be obtained from one or more of the aforementioned modalities, including acoustic, photoacoustic, piezoelectric, and optical.

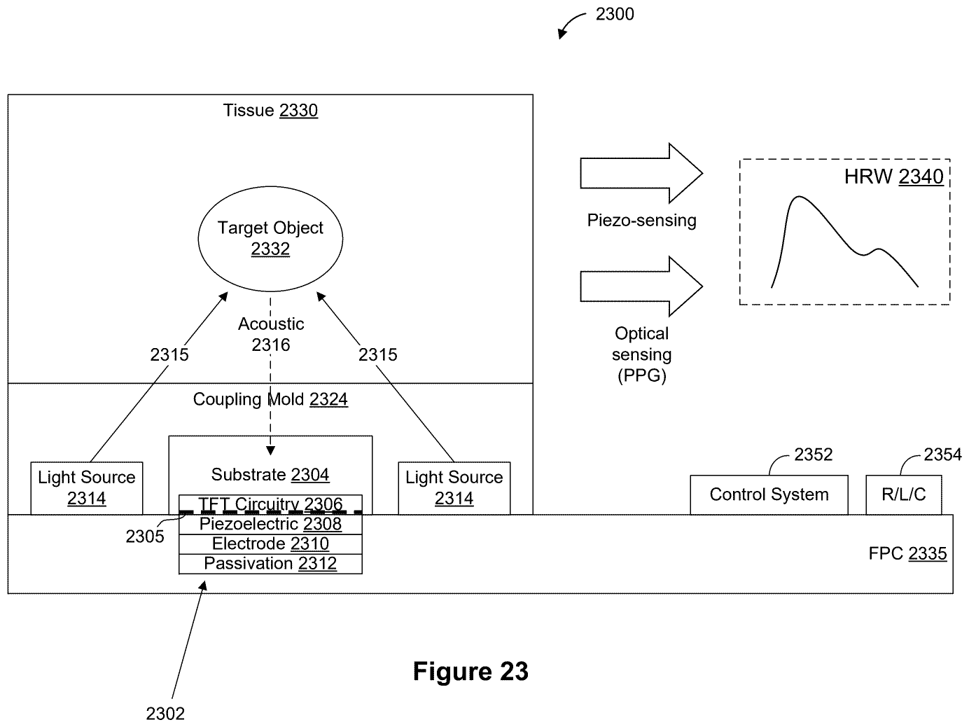

FIG. 23 is a cross-sectional diagram of another example implementation of a sensor apparatus 2300 with photoacoustics capability, according to some embodiments.

The sensor apparatus 2300 may be similar to sensor apparatus 2200. However, the sensor stack 2302 may be integrated in a coupling mold 2324, rather than secured or attached to the tissue 2330 via a coupling layer 2224. The coupling mold 2324 may be a malleable structure that shapes, aligns, or integrates components such as at least one component of the sensor stack 2302, ensuring mechanical, optical, and/or acoustic coupling between materials and media. For example, the coupling mold 2324 may be constructed of a transparent polymer or material such as silicone or PMMA.

In some implementations, a substrate 2304 and associated TFT circuitry 2306 (including one or more receiver pixels 2305) may be embedded or otherwise fixed within and held in the coupling mold 2324. The substrate 2304 may be an example of substrate 2204 and may thus be a flexible substrate composed of a flexible material such as polyimide or other flexible polymers. In some implementations, other components such as a piezoelectric layer 2308, an electrode layer 2310, and/or a passivation layer 2312 may be on or embedded an FPC 2335. However, in other implementations, some or all of the foregoing components may be in the coupling mold 2324 or the FPC 2335. A control system 2352 may be communicatively and electrically coupled to TFT circuitry 2306 and pixels 2305, as well as electrode layer 2310. One or more passive components 2354 may also be on board the FPC 2335.

In some embodiments, the sensor apparatus 2300 may include a light source system, which may include one or more light sources 2314. The one or more light sources 2314 may be examples of the one or more light sources 2214.

In some embodiments, the illustrated sensor apparatus 2300 may be configured to perform passive piezo-sensing and optical (PPG-based) sensing, similar to sensor apparatus 2200. Thus, HRW 2340 and heart rate waveform graphs 2118 may be captured using the sensor apparatus 2300.

### Example System Configurations

FIGS. 24A and 24B show block diagrams of example system configurations 2400 and 2420 of a sensor stack.

In some embodiments, as shown in FIG. 24A, a sensor element 2402 may be disposed on a flexible substrate 2404. The sensor element 2402 may be an example of acoustic sensing system 104 or sensing element 402, 502, 602 or 702, and may include at least some of the components and materials discussed above with respect according to various embodiments. The flexible substrate 2404 may be an example of flexible substrate 103 or substrate 404, 504, 604, 704, 904 or 1004.

In some embodiments, an active area 2403 may be associated with the sensor element 2402. In some configurations, the active area 2403 may correspond to an area from which acoustic signals (e.g., ultrasonic waves) may be emitted from one or more acoustic transmitter elements (e.g., an electrode layer or one or more electrode portions thereof, such as 410, or 410a, 410b and/or 410n), and/or where one or more acoustic receiver elements (e.g., one or more receiver pixels, such as 405) are disposed for detection of returning acoustic signals. In some implementations, the width of the active area 2403 may be at least a distance x, which may be the width of one or more of the layers in the various sensor stacks shown, e.g., in FIGS. 4, 4A, 5, 6, 7 and others. In some cases, other components such as peripheral circuits (e.g., column drivers, multiplexers) and/or interconnects and bond pads that allow interconnection between a circuit component and an electronic device may be included in (e.g., along the periphery) or outside of the active area 2403. For example, the active area 2403 may be electrically and/or communicatively coupled with the flexible substrate 2404.

In some embodiments, the flexible substrate 2404 may be electrically and/or communicatively coupled with a system 2406, e.g., on another substrate, which may be include a control system 2408 (e.g., an ASIC and/or a processor apparatus having one or more processors). In some implementations, control system 2408 may provide to the flexible substrate 2404 (including to one or more acoustic transmitter elements) transmit signals having a voltage at a fixed target frequency through a resonant circuit 2410 that may include one or more inductors (L1, L2) and/or a capacitor (C) (e.g., as discussed above with respect to FIG. 4). Control system 2408 may receive signals through a bus 2412 from the flexible substrate 2404 (including from one or more acoustic receiver elements), where the signals may be representative of returning acoustic signals. The received signals may be used for imaging, e.g., fingerprint imaging.

In some configurations, the control system 2408 may be configured to be communicative with another portion of a host device, e.g., a memory, another processor, a power source (e.g., battery). Different interfaces and protocols may be used. For example, a Serial Peripheral Interface (SPI) may be used to transmit the data back to the host device. An interrupt protocol (INTR) may be used to handle requests for the control system 2408 to interrupt currently executing instructions such that, e.g., imaging data can be collected, stop the current process to determine next commands, or process other events. A power interface (PWR) may be used to provide power to the control system 2408 and/or the flexible substrate 2404 (including the sensor element 2402).

In some embodiments, as shown in FIG. 24B, the sensor element 2402 and the control system 2408 may both be disposed on the flexible substrate 2404. In some implementations, control system 2408 may provide transmit signals directly to the sensor element 2402 through the resonant circuit 2410, which is on the same substrate. Control system 2408 may receive signals through the bus 2412 directly from the sensor element 2402.

Accordingly, it can be seen that only the sensor element 2402 may be on the flexible substrate 2404 in some configurations, or the sensor element 2402, the control system 2408 may be on the flexible substrate 2404 in some configurations.

In some cases, some or all the aforementioned example stacks of materials (e.g., 400, 500, 600, 700, 800, 900, 1000) may be implemented as a sensor, sensor stack, or a portion thereof, with various types of devices. For example, they may be used with flexible devices such as foldable displays or curved platens. Typically, a rigid or inflexible platen or other stable medium for acoustic (e.g., ultrasonic) signal paths and diffraction facilitates spatial resolution in sensor readings that are obtained as discussed throughout the present disclosure. Typically, ultrasound waves obey diffraction limits, which can restrict resolution. However, it has been found that even in some physically flexible implementations, high spatial resolution (e.g., 3 to 4 line pairs per millimeter, or more in some cases) may be obtained at the near-field region close to the sensor, even over temperature variations, as a result of contributing phenomena such as near-field super resolution imaging involving near-field interactions and non-linear effects, combined with a thin sensor stack having dimensions mentioned above. The example stacks of materials may operate in a near-field mode-operating with signal transmission close to the sensor (e.g., while a target object is pressed against a layer adjacent to the sensor stack)—or in a far-field mode.

Specifically, in some implementations, a sensor stack may be configured to transmit acoustic signals having ultra-low frequencies. In some examples, the sensor stack may transmit ultrasonic waves having a peak frequency of about 1-30 MHz (e.g., about 8 MHz). In some examples, the sensor stack may transmit ultrasonic waves having a peak frequency in the range from 1 MHz to 6 MHz. The frequency may be selected based on the display, passive components (e.g., LC circuit), and control system configuration, so as to match the display frequency.

Hence, the disclosed thin sensor stacks may be implemented in flexible devices and can advantageously produce high spatial resolution and imaging performance. Such flexible implementations can also be beneficial for enduring mechanical stresses such as fold-induced stresses.

### Example Methods

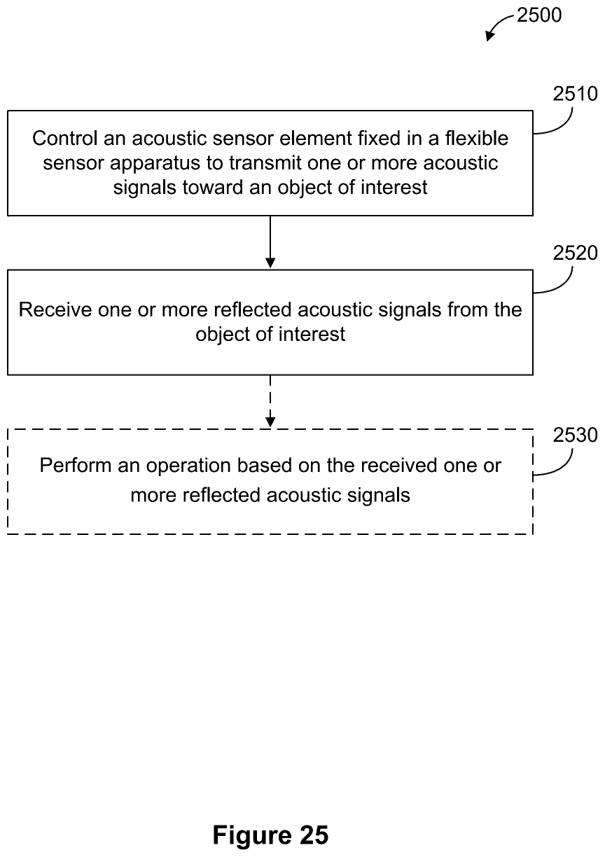

FIG. 25 is a flow diagram of an example of a method 2500 of operating a flexible acoustic sensor, according to some disclosed embodiments. Structure for performing the functionality illustrated in one or more of the blocks shown in FIG. 25 may be performed by hardware and/or software components, such as a control system, of an apparatus or system. Components of such apparatus or system may include, for example, an acoustic transmitter system, an acoustic receiver system, a control system (including one or more processors), a memory, and/or a computer-readable apparatus including a storage medium storing computer-readable and/or computer-executable instructions that are configured to, when executed by the control system, cause the control system, the one or more processors, or the apparatus or system to perform operations represented by blocks below. Example components of the apparatus or system are illustrated in, e.g., FIGS. 1, 4-12B and 14, which are described in more detail above.

The blocks of FIG. 25 may, for example, be performed by the apparatus 100 or by a similar apparatus, or a component thereof (e.g., a control system). The method outlined in FIG. 25 may include more or fewer blocks than indicated. Moreover, the blocks of methods disclosed herein are not necessarily performed in the order indicated. In some instances, one or more of the blocks shown in FIG. 25 may be performed concurrently.

At block 2510, the method 2500 may include controlling (e.g., by a control system, such as control system 106) an acoustic sensor element fixed in a flexible sensor apparatus to transmit one or more acoustic signals toward an object of interest (e.g., a finger of a user). In some cases, one or more acoustic signals may be ultrasonic signals transmitted by one or more acoustic transmitter elements.

In some scenarios, the flexible sensor apparatus may part of a flexible device such as a foldable device, and the foldable device may be in a deformed state during the transmission of the one or more acoustic signals. The deformed state may include two planes associated with the device intersecting. For example, deformed state of the device may include a folded state of the foldable device, where the device may be folded or bent such that one end of the device points in one direction while another end of the device points in another direction. It will be noted that more than two planes may be associated with the deformed state in some scenarios. For example, the device may be folded at multiple points or line segments such that there are three, four, or more planes associated with the device intersecting with one another (multiple corners may be folded, the device may be folded in a zig-zag fashion, there may be a curved surface or surfaces, etc.)

Means for performing functionality at block 2510 may include platen 101, acoustic transmitter system 104a, control system 106, and/or other components of the apparatus as shown in FIG. 1.

At block 2520, the method 2500 may include receiving one or more reflected acoustic signals from the object of interest. In some cases, the one or more reflected acoustic signals may be ultrasonic signals detected and received by one or more receiver elements, such as one or more receiver pixels, and the reflected acoustic signals may be representative of acoustic data, e.g., fingerprint data, from the imaging portion.

Means for performing functionality at block 2520 may include platen 101, acoustic receiver system 104b, and/or other components of the apparatus as shown in FIG. 1.

Some implementations of method 2500 may include, at block 2530, performing an operation based on the received one or more reflected acoustic signals. In some examples, acoustic data may be used to identify the object of interest or a portion thereof, generate imaging data (e.g., fingerprint imaging data) and/or an image based on the imaging data (e.g., fingerprint image), change an operative state of a device using the acoustic data, perform an operation with the device (initialize an application, display data, etc.), etc., or a combination thereof. In some examples, the operation may include determination of a physiological characteristic, such as pulse wave velocity (PWV), arterial stiffness, etc.

Means for performing functionality at block 2530 may include the control system 106 and/or other components of the apparatus as shown in FIG. 1.

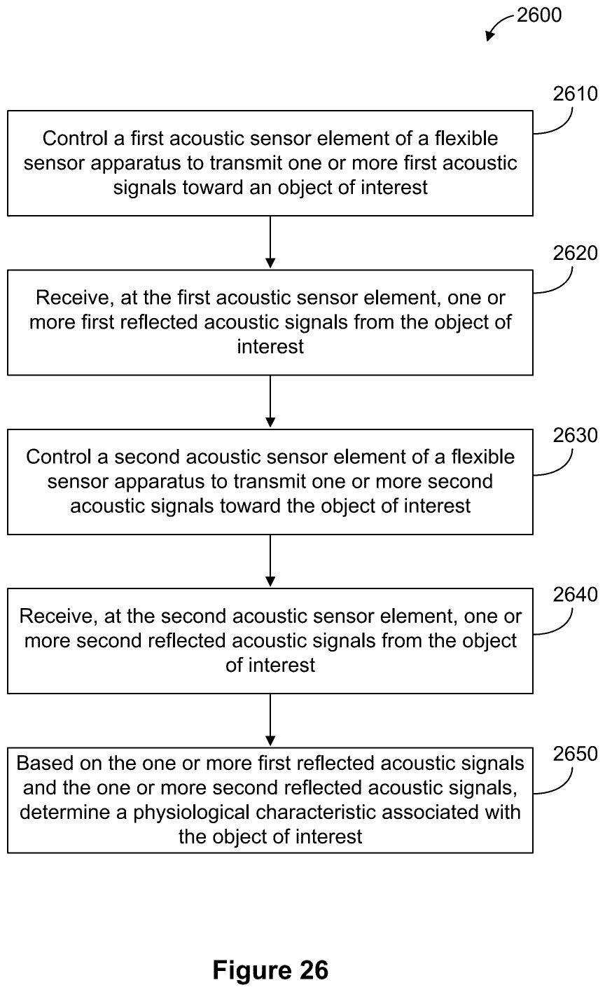

FIG. 26 is a flow diagram of another example of a method 2600 of operating a flexible acoustic sensor, according to some disclosed embodiments. Structure for performing the functionality illustrated in one or more of the blocks shown in FIG. 26 may be performed by hardware and/or software components, such as a control system, of an apparatus or system. Components of such apparatus or system may include, for example, an acoustic transmitter system, an acoustic receiver system, a control system (including one or more processors), a memory, and/or a computer-readable apparatus including a storage medium storing computer-readable and/or computer-executable instructions that are configured to, when executed by the control system, cause the control system, the one or more processors, or the apparatus or system to perform operations represented by blocks below. Example components of the apparatus or system are illustrated in, e.g., FIGS. 1, 4-12B and 14, which are described in more detail above.

The blocks of FIG. 26 may, for example, be performed by the apparatus 100 or by a similar apparatus, or a component thereof (e.g., a control system). The method outlined in FIG. 26 may include more or fewer blocks than indicated. Moreover, the blocks of methods disclosed herein are not necessarily performed in the order indicated. In some instances, one or more of the blocks shown in FIG. 26 may be performed concurrently.

At block 2610, the method 2600 may include controlling a first acoustic sensor element of a flexible sensor apparatus to transmit one or more first acoustic signals toward an object of interest. In some embodiments, the flexible acoustic sensor apparatus may include a flexible substrate, which may include a polymer such as polyimide; and thin-film transistor (TFT) circuitry comprising a plurality of pixelated sensor elements.

At block 2620, the method 2600 may include receiving, at the first acoustic sensor element, one or more first reflected acoustic signals from the object of interest.

At block 2630, the method 2600 may include controlling a second acoustic sensor element of a flexible sensor apparatus to transmit one or more second acoustic signals toward the object of interest.

At block 2640, the method 2600 may include receiving, at the second acoustic sensor element, one or more second reflected acoustic signals from the object of interest.

At block 2650, the method 2600 may include, based on the one or more first reflected acoustic signals and the one or more second reflected acoustic signals, determining a physiological characteristic associated with the object of interest. In some embodiments, the object of interest may include a blood vessel of a body part of a user. In some embodiments, the physiological characteristic may include pulse wave velocity (PWV) of the object of interest (e.g., blood vessel). In some approaches, the flexible acoustic sensor may be communicatively coupled with at least one control system, and the at least one control system may be configured to determine the PWV of the blood vessel based on a distance between the first acoustic sensor element and the second acoustic sensor element, and a time delay associated with the one or more first acoustic signals and the one or more second acoustic signals.

Means for performing functionality at blocks 2610-2650 may include acoustic transmitter system 104a, acoustic receiver system 104b, control system 106, and/or other components of the apparatus as shown in FIG. 1.

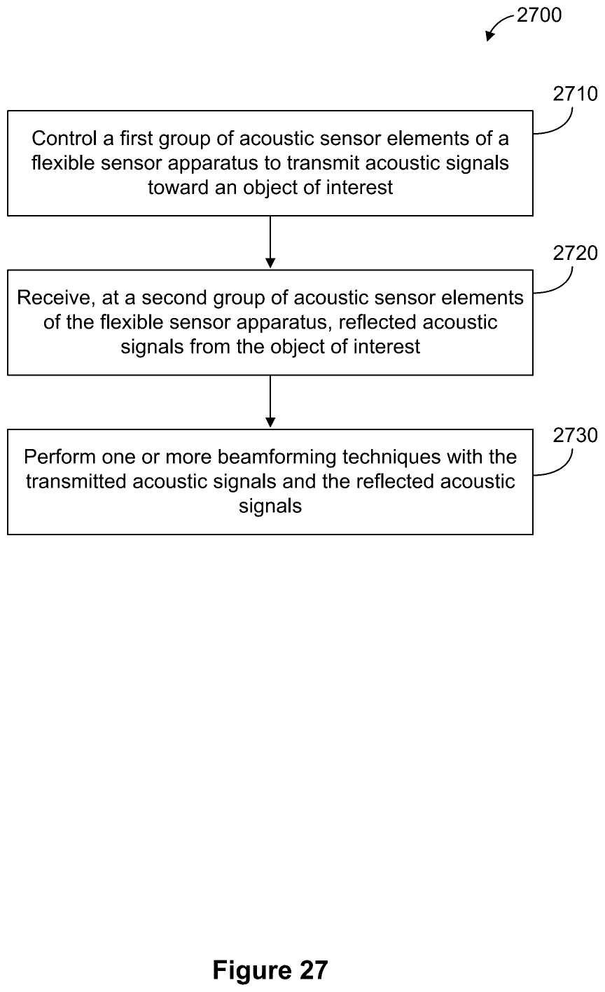

FIG. 27 is a flow diagram of another example of a method 2700 of operating a flexible acoustic sensor, according to some disclosed embodiments. Structure for performing the functionality illustrated in one or more of the blocks shown in FIG. 27 may be performed by hardware and/or software components, such as a control system, of an apparatus or system. Components of such apparatus or system may include, for example, an acoustic transmitter system, an acoustic receiver system, a control system (including one or more processors), a memory, and/or a computer-readable apparatus including a storage medium storing computer-readable and/or computer-executable instructions that are configured to, when executed by the control system, cause the control system, the one or more processors, or the apparatus or system to perform operations represented by blocks below. Example components of the apparatus or system are illustrated in, e.g., FIGS. 1, 4-12B and 14, which are described in more detail above.

The blocks of FIG. 27 may, for example, be performed by the apparatus 100 or by a similar apparatus, or a component thereof (e.g., a control system). The method outlined in FIG. 27 may include more or fewer blocks than indicated. Moreover, the blocks of methods disclosed herein are not necessarily performed in the order indicated. In some instances, one or more of the blocks shown in FIG. 27 may be performed concurrently.

At block 2710, the method 2700 may include controlling a first group of acoustic sensor elements of a flexible sensor apparatus to transmit acoustic signals toward an object of interest. In some embodiments, the flexible acoustic sensor apparatus may include: a flexible substrate comprising polyimide; and thin-film transistor (TFT) circuitry comprising a plurality of pixelated sensor elements. In some embodiments, the object of interest may include a blood vessel of a body part of a user.

At block 2720, the method 2700 may include receiving, at a second group of acoustic sensor elements of the flexible sensor apparatus, reflected acoustic signals from the object of interest. In some implementations, the first group of acoustic sensor elements may include a row of the plurality of pixelated sensor elements, and the second group of acoustic sensor elements may include a column of the plurality of pixelated sensor elements, the row and the column being substantially perpendicular to each other on the flexible acoustic sensor apparatus.

At block 2730, the method 2700 may include performing one or more beamforming techniques with the transmitted acoustic signals and the reflected acoustic signals. In some implementations, the one or more beamforming techniques may include: row-column driving based on the transmission of the acoustic signals by the first group of acoustic sensor elements and the receipt of the reflected acoustic signals by the second group of acoustic sensor elements; a delay-and-sum beamforming process comprising applying a time delay to one or more of the received reflected acoustic signals, and summing the received reflected acoustic signals; division of the plurality of pixelated sensor elements into subarrays; positioning at least a portion of the plurality of pixelated sensor elements at different heights; or a combination thereof.

In some approaches, the row-column driving may include: identifying one or more sensor elements from the plurality of pixelated sensor elements associated with higher received signal strength than other ones of the plurality of pixelated sensor elements; and activating a row and a column of the plurality of pixelated sensor elements associated with the identified one or more sensor elements. In some cases, the activated row may include the first group of acoustic sensor elements, and the activated column may include the second group of acoustic sensor elements. In some cases, the activated row may include the second group of acoustic sensor elements, and the activated column may include the first group of acoustic sensor elements.

Means for performing functionality at blocks 2710-2730 may include acoustic receiver system 104b, control system 106, and/or other components of the apparatus as shown in FIG. 1.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also may be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module which may reside on a computer-readable medium. Computer-readable media include both computer storage media and communication media including any medium that may be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those having ordinary skill in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “exemplary” is used exclusively herein, if at all, to mean “serving as an example, instance, or illustration.” Any implementation described herein as “exemplary” is not necessarily to be construed as preferred or advantageous over other implementations.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, other implementations are within the scope of the following claims. In some cases, the actions recited in the claims may be performed in a different order and still achieve desirable results.

It will be understood that unless features in any of the particular described implementations are expressly identified as incompatible with one another or the surrounding context implies that they are mutually exclusive and not readily combinable in a complementary and/or supportive sense, the totality of this disclosure contemplates and envisions that specific features of those complementary implementations may be selectively combined to provide one or more comprehensive, but slightly different, technical solutions. It will therefore be further appreciated that the above description has been given by way of example only and that modifications in detail may be made within the scope of this disclosure.

Various modifications to the implementations described in this disclosure may be readily apparent to those skilled in the art, and the generic principles defined herein may be applied to other implementations without departing from the spirit or scope of this disclosure. Thus, the following claims are not intended to be limited to the implementations shown herein, but are to be accorded the widest scope consistent with this disclosure, the principles and the novel features disclosed herein.

Additionally, certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. Moreover, various ones of the described and illustrated operations can itself include and collectively refer to a number of sub-operations. For example, each of the operations described above can itself involve the execution of a process or algorithm. Furthermore, various ones of the described and illustrated operations can be combined or performed in parallel in some implementations. Similarly, the separation of various system components in the implementations described above should not be understood as requiring such separation in all implementations. As such, other implementations are within the scope of the following claims. In some cases, the actions recited in the claims can be performed in a different order and still achieve desirable results.

Implementation examples are described in the following numbered clauses:
Clause 1: An acoustic sensing apparatus comprising: a flexible substrate comprising polyimide and having a thickness between 5 and 80 μm; and a flexible acoustic sensor element disposed adjacent to the flexible substrate, the flexible acoustic sensor element comprising a stack of materials, the stack of materials comprising: an acoustic receiver element configured to detect one or more acoustic signals received through the flexible substrate; a piezoelectric layer disposed adjacent to the acoustic receiver element; and an acoustic transmitter element configured to transmit one or more acoustic signals through the flexible substrate; wherein the flexible substrate and the flexible acoustic sensor element are configured to conform to a curvature of a surface that is constructed to contact a body part of a user from which the one or more acoustic signals transmitted from the acoustic transmitter element are reflected.
Clause 2: The acoustic sensing apparatus of clause 1, wherein the acoustic receiver element comprises one or more receiver pixels of thin-film transistor (TFT) circuitry on the piezoelectric layer.
Clause 3: The acoustic sensing apparatus of clause 1, wherein the acoustic transmitter element is further configured to transmit the one or more acoustic signals responsive to the body part of the user contacting the surface.
Clause 4: The acoustic sensing apparatus of clause 1, wherein the acoustic transmitter element comprises a first electrode layer having a thickness of up to 100 μm.
Clause 5: The acoustic sensing apparatus of clause 4, further comprising a second piezoelectric layer having a thickness between 5 and 30 μm, and a second electrode layer having a thickness of up to 100 μm.
Clause 6: The acoustic sensing apparatus of clause 1, wherein the surface is part of a platen of a device implementing the acoustic sensing apparatus.
Clause 7: The acoustic sensing apparatus of clause 6, wherein the flexible substrate comprising polyimide is disposed closer to the platen than the acoustic transmitter element, and the thickness of the flexible substrate is between 2 to 20 μm.
Clause 8: The acoustic sensing apparatus of clause 6, wherein the flexible substrate comprising polyimide is disposed farther from the platen than the acoustic transmitter element, and the thickness of the flexible substrate is between 30 to 70 μm.
Clause 9: The acoustic sensing apparatus of clause 8, further comprising a metallic or glass layer, disposed adjacent to the flexible substrate comprising polyimide and disposed opposite to the acoustic receiver element and the acoustic transmitter element.
Clause 10: The acoustic sensing apparatus of clause 1, wherein: the flexible acoustic sensor element is communicatively coupled with at least one control system that is disposed outside the flexible substrate; and the at least one control system is configured to provide a voltage to the flexible acoustic sensor element via a resonating circuit, the voltage causing the acoustic transmitter element to generate the one or more acoustic signals at a frequency of up to 30 MHz.
Clause 11: The acoustic sensing apparatus of clause 1, wherein: the flexible acoustic sensor element is communicatively coupled with at least one control system that is disposed on the flexible substrate; and the at least one control system is configured to provide a voltage to the flexible acoustic sensor element via a resonating circuit, the voltage causing the acoustic transmitter element to generate the one or more acoustic signals at a frequency of up to 30 MHz.
Clause 12: The acoustic sensing apparatus of clause 1, wherein the stack of materials further comprises a passivation layer having a thickness of up to 100 μm.
Clause 13: A flexible display apparatus comprising: a glass-based or plastic-based cover layer; a light-emitting layer disposed adjacent to the cover layer; and a flexible acoustic sensing element comprising: a polyimide substrate; an acoustic receiver element configured to detect one or more acoustic signals received through the polyimide substrate and the light-emitting layer; and an acoustic transmitter element configured to transmit one or more acoustic signals through the polyimide substrate and the light-emitting layer; wherein the flexible acoustic sensing element and the flexible display apparatus are configured to collectively deform such that at least two planes associated with the flexible display apparatus intersect one another during a deformed state of the flexible display apparatus.
Clause 14: The flexible display apparatus of clause 13, wherein the flexible acoustic sensing element is disposed within the flexible display apparatus and disposed adjacent to the light-emitting layer.
Clause 15: The flexible display apparatus of clause 13, wherein the flexible acoustic sensing element is laminated to the flexible display apparatus via an adhesive layer.
Clause 16: The flexible display apparatus of clause 13, wherein: the flexible display apparatus comprises a foldable device; and the deformed state of the flexible display apparatus comprises a folded state of the foldable device.
Clause 17: The flexible display apparatus of clause 13, wherein the light-emitting layer of the flexible display apparatus comprises an organic light-emitting diode (OLED) panel.
Clause 18: The flexible display apparatus of clause 13, wherein the acoustic receiver element comprises one or more pixelated receiver electrodes having associated thin-film transistor (TFT) circuitry.
Clause 19: An acoustic sensing apparatus comprising: a flexible substrate comprising polyimide and having a thickness between 5 and 80 μm; and a flexible acoustic sensor element disposed adjacent to the flexible substrate, the flexible acoustic sensor element comprising a stack of materials, the stack of materials comprising: an acoustic receiver element configured to detect one or more acoustic signals received through the flexible substrate; a first piezoelectric layer disposed adjacent to the acoustic receiver element; a first acoustic transmitter element configured to transmit one or more acoustic signals through the flexible substrate; and a second acoustic transmitter element configured to transmit one or more acoustic signals through the flexible substrate; wherein the flexible substrate and the flexible acoustic sensor element are configured to conform to a curvature of a surface that is constructed to contact a body part of a user from which the one or more acoustic signals transmitted from the first acoustic transmitter element and the second acoustic transmitter element are reflected.
Clause 20: The acoustic sensing apparatus of clause 19, wherein: the acoustic receiver element comprises one or more pixelated receiver electrodes having associated thin-film transistor (TFT) circuitry on the first piezoelectric layer; the first acoustic transmitter element comprises a first electrode layer having a thickness of up to 100 μm; the second acoustic transmitter element comprises a second electrode layer having a thickness of up to 100 μm; and at least one of the first electrode layer or the second electrode layer comprises conductive ink.

What is claimed:

1. An acoustic sensing apparatus comprising:
a flexible substrate comprising polyimide and having a thickness between 5 and 80 μm; and
a flexible acoustic sensor element disposed adjacent to the flexible substrate, the flexible acoustic sensor element comprising a stack of materials, the stack of materials comprising:
an acoustic receiver element configured to detect one or more acoustic signals received through the flexible substrate;
a piezoelectric layer disposed adjacent to the acoustic receiver element; and
an acoustic transmitter element configured to transmit one or more acoustic signals through the flexible substrate;
wherein the flexible substrate and the flexible acoustic sensor element are configured to conform to a curvature of a surface that is constructed to contact a body part of a user from which the one or more acoustic signals transmitted from the acoustic transmitter element are reflected.

2. The acoustic sensing apparatus of claim 1, wherein the acoustic receiver element comprises one or more receiver pixels of thin-film transistor (TFT) circuitry on the piezoelectric layer.

3. The acoustic sensing apparatus of claim 1, wherein the acoustic transmitter element is further configured to transmit the one or more acoustic signals responsive to the body part of the user contacting the surface.

4. The acoustic sensing apparatus of claim 1, wherein the acoustic transmitter element comprises a first electrode layer having a thickness of up to 100 μm.

5. The acoustic sensing apparatus of claim 4, further comprising a second piezoelectric layer having a thickness between 5 and 30 μm, and a second electrode layer having a thickness of up to 100 μm.

6. The acoustic sensing apparatus of claim 1, wherein the surface is part of a platen of a device implementing the acoustic sensing apparatus.

7. The acoustic sensing apparatus of claim 6, wherein the flexible substrate comprising polyimide is disposed closer to the platen than the acoustic transmitter element, and the thickness of the flexible substrate is between 2 to 20 μm.

8. The acoustic sensing apparatus of claim 6, wherein the flexible substrate comprising polyimide is disposed farther from the platen than the acoustic transmitter element, and the thickness of the flexible substrate is between 30 to 70 μm.

9. The acoustic sensing apparatus of claim 8, further comprising a metallic or glass layer, disposed adjacent to the flexible substrate comprising polyimide and disposed opposite to the acoustic receiver element and the acoustic transmitter element.

10. The acoustic sensing apparatus of claim 1, wherein:
the flexible acoustic sensor element is communicatively coupled with at least one control system that is disposed outside the flexible substrate; and
the at least one control system is configured to provide a voltage to the flexible acoustic sensor element via a resonating circuit, the voltage causing the acoustic transmitter element to generate the one or more acoustic signals at a frequency of up to 30 MHz.

11. The acoustic sensing apparatus of claim 1, wherein:
the flexible acoustic sensor element is communicatively coupled with at least one control system that is disposed on the flexible substrate; and
the at least one control system is configured to provide a voltage to the flexible acoustic sensor element via a resonating circuit, the voltage causing the acoustic transmitter element to generate the one or more acoustic signals at a frequency of up to 30 MHz.

12. The acoustic sensing apparatus of claim 1, wherein the stack of materials further comprises a passivation layer having a thickness of up to 100 μm.

13. A flexible display apparatus comprising:
a glass-based or plastic-based cover layer;
a light-emitting layer disposed adjacent to the cover layer; and
a flexible acoustic sensing element comprising:
a polyimide substrate;
an acoustic receiver element configured to detect one or more acoustic signals received through the polyimide substrate and the light-emitting layer; and
an acoustic transmitter element configured to transmit one or more acoustic signals through the polyimide substrate and the light-emitting layer;
wherein the flexible acoustic sensing element and the flexible display apparatus are configured to collectively deform such that at least two planes associated with the flexible display apparatus intersect one another during a deformed state of the flexible display apparatus.

14. The flexible display apparatus of claim 13, wherein the flexible acoustic sensing element is disposed within the flexible display apparatus and disposed adjacent to the light-emitting layer.

15. The flexible display apparatus of claim 13, wherein the flexible acoustic sensing element is laminated to the flexible display apparatus via an adhesive layer.

16. The flexible display apparatus of claim 13, wherein:
the flexible display apparatus comprises a foldable device; and
the deformed state of the flexible display apparatus comprises a folded state of the foldable device.

17. The flexible display apparatus of claim 13, wherein the light-emitting layer of the flexible display apparatus comprises an organic light-emitting diode (OLED) panel.

18. The flexible display apparatus of claim 13, wherein the acoustic receiver element comprises one or more pixelated receiver electrodes having associated thin-film transistor (TFT) circuitry.

19. An acoustic sensing apparatus comprising:
a flexible substrate comprising polyimide and having a thickness between 5 and 80 μm; and
a flexible acoustic sensor element disposed adjacent to the flexible substrate, the flexible acoustic sensor element comprising a stack of materials, the stack of materials comprising:
an acoustic receiver element configured to detect one or more acoustic signals received through the flexible substrate;
a first piezoelectric layer disposed adjacent to the acoustic receiver element;
a first acoustic transmitter element configured to transmit one or more acoustic signals through the flexible substrate; and
a second acoustic transmitter element configured to transmit one or more acoustic signals through the flexible substrate;
wherein the flexible substrate and the flexible acoustic sensor element are configured to conform to a curvature of a surface that is constructed to contact a body part of a user from which the one or more acoustic signals transmitted from the first acoustic transmitter element and the second acoustic transmitter element are reflected.

20. The acoustic sensing apparatus of claim 19, wherein:
the acoustic receiver element comprises one or more pixelated receiver electrodes having associated thin-film transistor (TFT) circuitry on the first piezoelectric layer;
the first acoustic transmitter element comprises a first electrode layer having a thickness of up to 100 μm;
the second acoustic transmitter element comprises a second electrode layer having a thickness of up to 100 μm; and
at least one of the first electrode layer or the second electrode layer comprises conductive ink.
